SEQUENCE LISTING (215 sequences)

SEQ ID NO: 1 - protein, 118 aa, Artificial Sequence
  2-C3 Heavy Chain Variable Region
EVQLVESGGDLVKPGGSLKLSCAASGFTFSSYGMSWVRQTPDKRLEWVATISGGGSYTYYLDSVKGRFTISRDIAKNTLYLQMSSLKSEDTAMYFCARQSRGNAMDYWGQGTSVTVSS

SEQ ID NO: 2 - protein, 113 aa, Artificial Sequence
  2-C3 Light Chain Variable Region
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 3 - protein, 117 aa, Artificial Sequence
  2-P8 Heavy Chain Variable Region
EVQLQQSGPELVKPGASVKMSCKASGYSFTGYNMHWVKQSHGKSLEWIGYIDPYNGVTNYNQKFKGKATLTVDKSSSTAYVQLNSLTSEDSAVYYCARWGGNYVDYWGQGTTLKVSS

SEQ ID NO: 4 - protein, 107 aa, Artificial Sequence
  2-P8 Light Chain Variable Region
DIKMTQSPSSMYASLGERVTITCKASQDINRYLSWFQQKPGKSPKTLIYRANRLVDGVPSRFSGSFSGQDYSLTISSLEYEDMGIYYCLQYDEFPLTFGAGTKLELK

SEQ ID NO: 5 - protein, 118 aa, Artificial Sequence
  3-E21 Heavy Chain Variable Region
EVQLVESGGALVKPGGSLKLSCAASGFTFSKYAMSWVRQTPEKRLEWVAFISNGGSYTYCLDSVKGRFTISRDNAKNTLYLQMSSLRSEDTALYYCARHDKGNALDYWGQGNSVTVSS

SEQ ID NO: 6 - protein, 113 aa, Artificial Sequence
  3-E21 Light Chain Variable Region
DIVMTQSPSSLTVTAGEKVTMSCKSSQSLLNSGNQKNYLTWYQQKPGQPPKLLIYWASTRESGVPDRFTGSGSGTDFTLTISSVQAEDLSVYYCQNDYFYPLTFGAGTKLELK

SEQ ID NO: 7 - protein, 119 aa, Artificial Sequence
  3-P21 Heavy Chain Variable Region
EIQLQQSGAELVKPGASVKISCKASGYSFTGYNMKWVKQSHGKSLEWIGNINPYFGSTNYNQKFKGKATLTVDKSSSTAYMQLNSLTSEDSAVYYCARGAYYGNAMDYWGQGTSVTVSS

SEQ ID NO: 8 - protein, 113 aa, Artificial Sequence
  3-P21 Light Chain Variable Region
DIVMTQSPSSLTVTAGGKVTMSCKSSQSLLNSGNQKNYLTWYQQKPGQPPKLLIYWASTRESGVPDRFSGSGSGTDFTLTISSVQAEDLAVYYCQNDYFYPLTFGAGTKLELK

SEQ ID NO: 9 - protein, 118 aa, Artificial Sequence
  5-E22 Heavy Chain Variable Region
KVQLQQSGPDLVEPGASVKISCKASGYTITDNYMHWVKQKPGQGLEWIGEIYPGSGNTYYNERFKGKATLTADKSSSTAYMQLSSLTSEDSAVYFCARGFPYYAMDYWGPGTSVTVSS

SEQ ID NO: 10 - protein, 107 aa, Artificial Sequence
  5-E22 Light Chain Variable Region
DIQMNQSPSSLSASLGDTITITCHARQNINVWLSWYQQKSGNIPKLLIYKASNLHTGVPSRFSGSGSGTRFTLTISSLQPEDMATYYCQQGQNYPLTFGGGTKLEIK

SEQ ID NO: 11 - protein, 119 aa, Artificial Sequence
  6-J11 Heavy Chain Variable Region
DVQLVESGGGLVQPGGSRKLSCAASGFIFSSFGMHWVRQAPEKGLEWVAYISSGRSTMYYADTVKGRFTISRDNPKNTLFLQMTSLRSEDTAMYYCARGGFYGNSLDYWGQGTSVTVSS

SEQ ID NO: 12 - protein, 113 aa, Artificial Sequence
  6-J11 Light Chain Variable Region
DIVMTQSPSSLTVTAGEKVTMSCKSSLSLLNSGNQKNYLTWYQQKPGQPPKLLIYWASTRESGVPDRFTGSGSGTDFTLTISSMQAEDLAVYSCQNAYSYPLTFGAGTKLELK

SEQ ID NO: 13 - protein, 119 aa, Artificial Sequence
  8-G12 Heavy Chain Variable Region
QVQLQQSGPELVKPGASVKISCKASGYAFSDYWMNWVKQRPGKGLEWIGQIYPGYGDTKYNENFKGTATLTADKSSSTAYMQLSSLTSEDSAVYFCARWGYYGNAMDYWGQGTSVTVSS

SEQ ID NO: 14 - protein, 113 aa, Artificial Sequence
  8-G12 Light Chain Variable Region
DIVMTQSPSSLTVTAGEKVTMSCKSSQSLLNSGNQKNYLTWYQQKPGQPPKLLIYWASTRESGVPDRFTGSGSGTDFTLTISSVQTEDLAIYYCQNAYIYPLTFGAGTKLELK

SEQ ID NO: 15 - protein, 118 aa, Artificial Sequence
  10-J10 Heavy Chain Variable Region
QVQLQQPGAELVKPGASVKLSCKASGYTFTRYRMNWVKQRPGQGLEWIGNIDPSDSETHYNQKFKDKATLTVDKSSSTAYMQLSSLTSEDSAVFYCARLNYGNCFDYWGQGTTLTVSS

SEQ ID NO: 16 - protein, 113 aa, Artificial Sequence
  10-J10 Light Chain Variable Region
DIVMTQSPSSLTVTAGEKVTMSCKSSQTLLNSGNQKNYLTWYQQKPGQPPKLLIYWASTRESGVPDRFTGSGSGTDFTLTISSVQAEDLAVFYCQNDYFYPFTFGSGTKLEIK

SEQ ID NO: 17 - protein, 118 aa, Artificial Sequence
  10-K2 Heavy Chain Variable Region
EVQLQQSGPELVKPGASVKMSCKASGYAFTSYVMHWVKQKPGQGLEWIGYINPYSDGTRYNEKFKGKATLTSDKSSSTAYMELSSLTSEDSAVYYCTRIYYGNAMDYWGQGTSVTVSS

SEQ ID NO: 18 - protein, 113 aa, Artificial Sequence
  10-K2 Light Chain Variable Region
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 19 - protein, 118 aa, Artificial Sequence
  15-D6 Heavy Chain Variable Region
QVQLQQPGADLVKPGASVKLSCKASGYTFTSYWINWVKQRPGQGLEWIGNIYPGRSSTNYNEKFKSKATLTVDTSSSTAYMQLSSLASDDSAVYYCSRLSRGNAMDYWGQGTSVTVSS

SEQ ID NO: 20 - protein, 113 aa, Artificial Sequence
  15-D6 Light Chain Variable Region
DIVMTQSPSSLTVTAGEKVTMSCRSSQSLLNSGNQKSYLTWYQQKPGQPPKLLIYWASTRESGVPDRFTGSGSGTDFTLTISSVQAEDLAVYYCQNDYYYPFTFGSGTKLEIK

SEQ ID NO: 21 - protein, 8 aa, Artificial Sequence
  2-C3 HC CDR1
GFTFSSYG

SEQ ID NO: 22 - protein, 8 aa, Artificial Sequence
  2-C3 HC CDR2
ISGGGSYT

SEQ ID NO: 23 - protein, 11 aa, Artificial Sequence
  2-C3 HC CDR3
ARQSRGNAMDY

SEQ ID NO: 24 - protein, 8 aa, Artificial Sequence
  2-P8 HC CDR1
GYSFTGYN

SEQ ID NO: 25 - protein, 8 aa, Artificial Sequence
  2-P8 HC CDR2
IDPYNGVT

SEQ ID NO: 26 - protein, 10 aa, Artificial Sequence
  2-P8 HC CDR3
ARWGGNYVDY

SEQ ID NO: 27 - protein, 8 aa, Artificial Sequence
  3-E21 HC CDR1
GFTFSKYA

SEQ ID NO: 28 - protein, 8 aa, Artificial Sequence
  3-E21 HC CDR2
ISNGGSYT

SEQ ID NO: 29 - protein, 11 aa, Artificial Sequence
  3-E21 HC CDR3
ARHDKGNALDY

SEQ ID NO: 30 - protein, 8 aa, Artificial Sequence
  3-P21 HC CDR1
GYSFTGYN

SEQ ID NO: 31 - protein, 8 aa, Artificial Sequence
  3-P21 HC CDR2
INPYFGST

SEQ ID NO: 32 - protein, 12 aa, Artificial Sequence
  3-P21 HC CDR3
ARGAYYGNAMDY

SEQ ID NO: 33 - protein, 8 aa, Artificial Sequence
  5-E22 HC CDR1
GYTITDNY

SEQ ID NO: 34 - protein, 8 aa, Artificial Sequence
  5-E22 HC CDR2
IYPGSGNT

SEQ ID NO: 35 - protein, 11 aa, Artificial Sequence
  5-E22 HC CDR3
ARGFPYYAMDY

SEQ ID NO: 36 - protein, 8 aa, Artificial Sequence
  6-J11 HC CDR1
GFIFSSFG

SEQ ID NO: 37 - protein, 8 aa, Artificial Sequence
  6-J11 HC CDR2
ISSGRSTM

SEQ ID NO: 38 - protein, 12 aa, Artificial Sequence
  6-J11 HC CDR3
ARGGFYGNSLDY

SEQ ID NO: 39 - protein, 8 aa, Artificial Sequence
  8-G12 HC CDR1
GYAFSDYW

SEQ ID NO: 40 - protein, 8 aa, Artificial Sequence
  8-G12 HC CDR2
IYPGYGDT

SEQ ID NO: 41 - protein, 12 aa, Artificial Sequence
  8-G12 HC CDR3
ARWGYYGNAMDY

SEQ ID NO: 42 - protein, 8 aa, Artificial Sequence
  10-J10 HC CDR1
GYTFTRYR

SEQ ID NO: 43 - protein, 8 aa, Artificial Sequence
  10-J10 HC CDR2
IDPSDSET

SEQ ID NO: 44 - protein, 11 aa, Artificial Sequence
  10-J10 HC CDR3
ARLNYGNCFDY

SEQ ID NO: 45 - protein, 8 aa, Artificial Sequence
  10-K2 HC CDR1
GYAFTSYV

SEQ ID NO: 46 - protein, 8 aa, Artificial Sequence
  10-K2 HC CDR2
INPYSDGT

SEQ ID NO: 47 - protein, 11 aa, Artificial Sequence
  10-K2 HC CDR3
TRIYYGNAMDY

SEQ ID NO: 48 - protein, 8 aa, Artificial Sequence
  15-D6 HC CDR1
GYTFTSYW

SEQ ID NO: 49 - protein, 8 aa, Artificial Sequence
  15-D6 HC CDR2
IYPGRSST

SEQ ID NO: 50 - protein, 11 aa, Artificial Sequence
  15-D6 HC CDR3
SRLSRGNAMDY

SEQ ID NO: 51 - protein, 12 aa, Artificial Sequence
  2-C3 LC CDR1
QSLLNSGNQKNY

SEQ ID NO: 52 - protein, 3 aa, Artificial Sequence
  2-C3 LC CDR2
WAS

SEQ ID NO: 53 - protein, 9 aa, Artificial Sequence
  2-C3 LC CDR3
QNDYSYPLT

SEQ ID NO: 54 - protein, 6 aa, Artificial Sequence
  2-P8 LC CDR1
QDINRY

SEQ ID NO: 55 - protein, 3 aa, Artificial Sequence
  2-P8 LC CDR2
RAN

SEQ ID NO: 56 - protein, 9 aa, Artificial Sequence
  2-P8 LC CDR3
LQYDEFPLT

SEQ ID NO: 57 - protein, 12 aa, Artificial Sequence
  3-E21 LC CDR1
QSLLNSGNQKNY

SEQ ID NO: 58 - protein, 3 aa, Artificial Sequence
  3-E21 LC CDR2
WAS

SEQ ID NO: 59 - protein, 9 aa, Artificial Sequence
  3-E21 LC CDR3
QNDYFYPLT

SEQ ID NO: 60 - protein, 12 aa, Artificial Sequence
  3-P21 LC CDR1
QSLLNSGNQKNY

SEQ ID NO: 61 - protein, 3 aa, Artificial Sequence
  3-P21 LC CDR2
WAS

SEQ ID NO: 62 - protein, 9 aa, Artificial Sequence
  3-P21 LC CDR3
QNDYFYPLT

SEQ ID NO: 63 - protein, 6 aa, Artificial Sequence
  5-E22 LC CDR1
QNINVW

SEQ ID NO: 64 - protein, 3 aa, Artificial Sequence
  5-E22 LC CDR2
KAS

SEQ ID NO: 65 - protein, 9 aa, Artificial Sequence
  5-E22 LC CDR3
QQGQNYPLT

SEQ ID NO: 66 - protein, 12 aa, Artificial Sequence
  6-J11 LC CDR1
LSLLNSGNQKNY

SEQ ID NO: 67 - protein, 3 aa, Artificial Sequence
  6-J11 LC CDR2
WAS

SEQ ID NO: 68 - protein, 9 aa, Artificial Sequence
  6-J11 LC CDR3
QNAYSYPLT

SEQ ID NO: 69 - protein, 12 aa, Artificial Sequence
  8-G12 LC CDR1
QSLLNSGNQKNY

SEQ ID NO: 70 - protein, 3 aa, Artificial Sequence
  8-G12 LC CDR2
WAS

SEQ ID NO: 71 - protein, 9 aa, Artificial Sequence
  8-G12 LC CDR3
QNAYIYPLT

SEQ ID NO: 72 - protein, 12 aa, Artificial Sequence
  10-J10 LC CDR1
QTLLNSGNQKNY

SEQ ID NO: 73 - protein, 3 aa, Artificial Sequence
  10-J10 LC CDR2
WAS

SEQ ID NO: 74 - protein, 9 aa, Artificial Sequence
  10-J10 LC CDR3
QNDYFYPFT

SEQ ID NO: 75 - protein, 12 aa, Artificial Sequence
  10-K2 LC CDR1
QSLLNSGNQKNY

SEQ ID NO: 76 - protein, 3 aa, Artificial Sequence
  10-K2 LC CDR2
WAS

SEQ ID NO: 77 - protein, 9 aa, Artificial Sequence
  10-K2 LC CDR3
QNDYSYPFT

SEQ ID NO: 78 - protein, 12 aa, Artificial Sequence
  15-D6 LC CDR1
QSLLNSGNQKSY

SEQ ID NO: 79 - protein, 3 aa, Artificial Sequence
  15-D6 LC CDR2
WAS

SEQ ID NO: 80 - protein, 9 aa, Artificial Sequence
  15-D6 LC CDR3
QNDYYYPFT

SEQ ID NO: 81 - protein, 10 aa, Artificial Sequence
  2-C3 HC CDR1
GFTFSSYGMS

SEQ ID NO: 82 - protein, 17 aa, Artificial Sequence
  2-C3 HC CDR2
TISGGGSYTYYLDSVKG

SEQ ID NO: 83 - protein, 11 aa, Artificial Sequence
  2-C3 HC CDR3
ARQSRGNAMDY

SEQ ID NO: 84 - protein, 10 aa, Artificial Sequence
  2-P8 HC CDR1
GYSFTGYNMH

SEQ ID NO: 85 - protein, 17 aa, Artificial Sequence
  2-P8 HC CDR2
YIDPYNGVTNYNQKFKG

SEQ ID NO: 86 - protein, 10 aa, Artificial Sequence
  2-P8 HC CDR3
ARWGGNYVDY

SEQ ID NO: 87 - protein, 10 aa, Artificial Sequence
  3-E21 HC CDR1
GFTFSKYAMS

SEQ ID NO: 88 - protein, 17 aa, Artificial Sequence
  3-E21 HC CDR2
FISNGGSYTYCLDSVKG

SEQ ID NO: 89 - protein, 11 aa, Artificial Sequence
  3-E21 HC CDR3
ARHDKGNALDY

SEQ ID NO: 90 - protein, 10 aa, Artificial Sequence
  3-P21 HC CDR1
GYSFTGYNMK

SEQ ID NO: 91 - protein, 17 aa, Artificial Sequence
  3-P21 HC CDR2
NINPYFGSTNYNQKFKG

SEQ ID NO: 92 - protein, 12 aa, Artificial Sequence
  3-P21 HC CDR3
ARGAYYGNAMDY

SEQ ID NO: 93 - protein, 10 aa, Artificial Sequence
  5-E22 HC CDR1
GYTITDNYMH

SEQ ID NO: 94 - protein, 17 aa, Artificial Sequence
  5-E22 HC CDR2
EIYPGSGNTYYNERFKG

SEQ ID NO: 95 - protein, 11 aa, Artificial Sequence
  5-E22 HC CDR3
ARGFPYYAMDY

SEQ ID NO: 96 - protein, 10 aa, Artificial Sequence
  6-J11 HC CDR1
GFIFSSFGMH

SEQ ID NO: 97 - protein, 17 aa, Artificial Sequence
  6-J11 HC CDR2
YISSGRSTMYYADTVKG

SEQ ID NO: 98 - protein, 12 aa, Artificial Sequence
  6-J11 HC CDR3
ARGGFYGNSLDY

SEQ ID NO: 99 - protein, 10 aa, Artificial Sequence
  8-G12 HC CDR1
GYAFSDYWMN

SEQ ID NO: 100 - protein, 17 aa, Artificial Sequence
  8-G12 HC CDR2
QIYPGYGDTKYNENFKG

SEQ ID NO: 101 - protein, 12 aa, Artificial Sequence
  8-G12 HC CDR3
ARWGYYGNAMDY

SEQ ID NO: 102 - protein, 10 aa, Artificial Sequence
  10-J10 HC CDR1
GYTFTRYRMN

SEQ ID NO: 103 - protein, 17 aa, Artificial Sequence
  10-J10 HC CDR2
NIDPSDSETHYNQKFKD

SEQ ID NO: 104 - protein, 11 aa, Artificial Sequence
  10-J10 HC CDR3
ARLNYGNCFDY

SEQ ID NO: 105 - protein, 10 aa, Artificial Sequence
  10-K2 HC CDR1
GYAFTSYVMH

SEQ ID NO: 106 - protein, 17 aa, Artificial Sequence
  10-K2 HC CDR2
YINPYSDGTRYNEKFKG

SEQ ID NO: 107 - protein, 11 aa, Artificial Sequence
  10-K2 HC CDR3
TRIYYGNAMDY

SEQ ID NO: 108 - protein, 10 aa, Artificial Sequence
  15-D6 HC CDR1
GYTFTSYWIN

SEQ ID NO: 109 - protein, 17 aa, Artificial Sequence
  15-D6 HC CDR2
NIYPGRSSTNYNEKFKS

SEQ ID NO: 110 - protein, 11 aa, Artificial Sequence
  15-D6 HC CDR3
SRLSRGNAMDY

SEQ ID NO: 111 - protein, 17 aa, Artificial Sequence
  2-C3 LC CDR1
KSSQSLLNSGNQKNYLT

SEQ ID NO: 112 - protein, 7 aa, Artificial Sequence
  2-C3 LC CDR2
WASTRES

SEQ ID NO: 113 - protein, 9 aa, Artificial Sequence
  2-C3 LC CDR3
QNDYSYPLT

SEQ ID NO: 114 - protein, 11 aa, Artificial Sequence
  2-P8 LC CDR1
KASQDINRYLS

SEQ ID NO: 115 - protein, 7 aa, Artificial Sequence
  2-P8 LC CDR2
RANRLVD

SEQ ID NO: 116 - protein, 9 aa, Artificial Sequence
  2-P8 LC CDR3
LQYDEFPLT

SEQ ID NO: 117 - protein, 17 aa, Artificial Sequence
  3-E21 LC CDR1
KSSQSLLNSGNQKNYLT

SEQ ID NO: 118 - protein, 7 aa, Artificial Sequence
  3-E21 LC CDR2
WASTRES

SEQ ID NO: 119 - protein, 9 aa, Artificial Sequence
  3-E21 LC CDR3
QNDYFYPLT

SEQ ID NO: 120 - protein, 17 aa, Artificial Sequence
  3-P21 LC CDR1
KSSQSLLNSGNQKNYLT

SEQ ID NO: 121 - protein, 7 aa, Artificial Sequence
  3-P21 LC CDR2
WASTRES

SEQ ID NO: 122 - protein, 9 aa, Artificial Sequence
  3-P21 LC CDR3
QNDYFYPLT

SEQ ID NO: 123 - protein, 11 aa, Artificial Sequence
  5-E22 LC CDR1
HARQNINVWLS

SEQ ID NO: 124 - protein, 7 aa, Artificial Sequence
  5-E22 LC CDR2
KASNLHT

SEQ ID NO: 125 - protein, 9 aa, Artificial Sequence
  5-E22 LC CDR3
QQGQNYPLT

SEQ ID NO: 126 - protein, 17 aa, Artificial Sequence
  6-J11 LC CDR1
KSSLSLLNSGNQKNYLT

SEQ ID NO: 127 - protein, 7 aa, Artificial Sequence
  6-J11 LC CDR2
WASTRES

SEQ ID NO: 128 - protein, 9 aa, Artificial Sequence
  6-J11 LC CDR3
QNAYSYPLT

SEQ ID NO: 129 - protein, 17 aa, Artificial Sequence
  8-G12 LC CDR1
KSSQSLLNSGNQKNYLT

SEQ ID NO: 130 - protein, 7 aa, Artificial Sequence
  8-G12 LC CDR2
WASTRES

SEQ ID NO: 131 - protein, 9 aa, Artificial Sequence
  8-G12 LC CDR3
QNAYIYPLT

SEQ ID NO: 132 - protein, 17 aa, Artificial Sequence
  10-J10 LC CDR1
KSSQTLLNSGNQKNYLT

SEQ ID NO: 133 - protein, 7 aa, Artificial Sequence
  10-J10 LC CDR2
WASTRES

SEQ ID NO: 134 - protein, 9 aa, Artificial Sequence
  10-J10 LC CDR3
QNDYFYPFT

SEQ ID NO: 135 - protein, 17 aa, Artificial Sequence
  10-K2 LC CDR1
KSSQSLLNSGNQKNYLT

SEQ ID NO: 136 - protein, 7 aa, Artificial Sequence
  10-K2 LC CDR2
WASTRES

SEQ ID NO: 137 - protein, 9 aa, Artificial Sequence
  10-K2 LC CDR3
QNDYSYPFT

SEQ ID NO: 138 - protein, 17 aa, Artificial Sequence
  15-D6 LC CDR1
RSSQSLLNSGNQKSYLT

SEQ ID NO: 139 - protein, 7 aa, Artificial Sequence
  15-D6 LC CDR2
WASTRES

SEQ ID NO: 140 - protein, 9 aa, Artificial Sequence
  15-D6 LC CDR3
QNDYYYPFT

SEQ ID NO: 141 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 142 - protein, 118 aa, Artificial Sequence
  2-C3-H1 Heavy Chain Variable Region
QVTLRESGPALVKPTQTLTLTCTASGFTFSSYGMSWVRQPPGKALEWVATISGGGSYTYYNPSLKDRFTISRDISANQLVLKVTNMDPADTATYFCARQSRGNAMDYWGQGTTVTVSS

SEQ ID NO: 143 - protein, 118 aa, Artificial Sequence
  2-C3-H2 Heavy Chain Variable Region
QVTLRESGPALVKPTQTLTLTCTFSGFTFSSYGMSWIRQPPGKALEWLATISGGGSYTYYLDSLKDRFTISRDISKNQVVLTVTNMDPADTATYFCARQSRGNAMDYWGQGTTVTVSS

SEQ ID NO: 144 - protein, 113 aa, Artificial Sequence
  2-C3-L1 Light Chain Variable Region
DIQMTQSPSTLSASVGDRVTITCKSSQSLLNSGNQKNYLTWYQQKPGKAPKLLIYWASTRESGVPSRFSGSGSGTAFTLTISSLQPDDFATYYCQNDYSYPLTFGGGTKVEIK

SEQ ID NO: 145 - protein, 113 aa, Artificial Sequence
  2-C3-L2 Light Chain Variable Region
DIQMTQSPSTLSASVGDRVTITCKSSQSLLNSGNQKNYLTWYQQKPGKAPKLLIYWASTRESGVPSRFSGSGSGTEFTLTISSLQPDDFATYYCQNDYSYPLTFGGGTKVEIK

SEQ ID NO: 146 - protein, 118 aa, Artificial Sequence
  5-E22-H1 Heavy Chain Variable Region
QVQLVQSGVEVKKPGASVKVSCKASGYTITDNYMHWVRQAPGQGLEWIGEIYPGSGNTYFNEKFKNRATLTADKSTTTAYMELKSLQFDDTAVYFCARGFPYYAMDYWGQGTTVTVSS

SEQ ID NO: 147 - protein, 118 aa, Artificial Sequence
  5-E22-H3 Heavy Chain Variable Region
QVQLVQSGAEVKKPGASVKVSCKASGYTITDNYMHWVRQAPGQGLEWIGEIYPGSGNTYYAEKFKNRATLTADKSISTAYMELSRLRSDDTAVYFCARGFPYYAMDYWGQGTLVTVSS

SEQ ID NO: 148 - protein, 107 aa, Artificial Sequence
  5-E22-L1 Light Chain Variable Region
EIVMTQSPATLSLSPGERATLSCHARQNINVWLSWYQQKPGQAPRLLIYKASNLHTGVPARFSGSGSGTDFTLTISSLEPEDFAVYYCQQGQNYPLTFGGGTKVEIK

SEQ ID NO: 149 - protein, 107 aa, Artificial Sequence
  5-E22-L2 Light Chain Variable Region
EIVLTQSPATLSLSPGERATLSCHARQNINVWLSWYQQKPGQAPRLLIYKASNLHTGIPARFSGSGSGTDFTLTISSLEPEDFAVYYCQQGQNYPLTFGGGTKVEIK

SEQ ID NO: 150 - protein, 107 aa, Artificial Sequence
  5-E22-L3 Light Chain Variable Region
DIVMTQSPLSLPVTPGEPASISCHARQNINVWLSWYLQKPGQSPQLLIYKASNLHTGVPDRFSGSGSGTDFTLKISRVEAEDVGVYYCQQGQNYPLTFGQGTKVEIK

SEQ ID NO: 151 - protein, 119 aa, Artificial Sequence
  6-J11-H1 Heavy Chain Variable Region
EVQLVESGGGLVQPGGSLRLSCAASGFIFSSFGMHWVRQAPGKGLEWVAYISSGRSTMYYADSVKGRFTISRDNSKNTLYLQMNSLTAEDTAVYYCARGGFYGNSLDYWGQGTLVTVSS

SEQ ID NO: 152 - protein, 119 aa, Artificial Sequence
  6-J11-H2 Heavy Chain Variable Region
EVQLVESGGGLVQPGGSLRLSCAASGFIFSSFGMHWVRQAPGKGLEWVAYISSGRSTMYYADSVKGRFTISRDNSKNTLYLQMNSLRSEDTAVYYCARGGFYGNSLDYWGQGTLVTVSS

SEQ ID NO: 153 - protein, 113 aa, Artificial Sequence
  6-J11-L1 Light Chain Variable Region
DIQMTQSPSSLSASVGDRVTITCKSSLSLLNSGNQKNYLTWYQQKPGKAPKLLIYWASTRESGVPSRFSGSGSGTDFTLTISSLQPEDFATYSCQNAYSYPLTFGQGTKVEIK

SEQ ID NO: 154 - protein, 118 aa, Artificial Sequence
  3-E21-H1 Heavy Chain Variable Region
QVQLQESGPGLVRPSQTLSLTCTASGFTFSKYAMNWVRQPPGRGLEWVAFISNGGSYTEYNPSVKGRFTILRDNSKNQLSLRLSSVTAADTAVYYCARHDKGNALDYWGQGSLVTVSS

SEQ ID NO: 155 - protein, 118 aa, Artificial Sequence
  3-E21-H2 Heavy Chain Variable Region
QVQLQESGPGLVRPSQTLSLTCTASGFTFSKYAMSWVRQPPGRGLEWVAFISNGGSYTEYNPSVKGRFTILRDNSKNQLSLKLSSVTAADTAVYYCARHDKGNALDYWGQGSLVTVSS

SEQ ID NO: 156 - protein, 118 aa, Artificial Sequence
  3-E21-H3 Heavy Chain Variable Region
EVQLLESGGGLVQPGGSLRLSCAASGFTFSKYAMSWVRQAPGKGLEWVAAISNGGSYTYYADSVKGRFTISRDNSKNTLYLQMNSLRAEDTAVYYCARHDKGNALDYWGQGTLVTVSS

SEQ ID NO: 157 - protein, 113 aa, Artificial Sequence
  3-E21-L1 Light Chain Variable Region
DIQMTQSPSSLSASVGDRVTITCKSSQSLLNSGNQKNYLTWYQQKPGKAPKLLIYWASNLQTGVPSRFSGSGSGTDFTFTISSLQPEDIATYYCQNDYFYPLTFGQGTKVEIK

SEQ ID NO: 158 - protein, 113 aa, Artificial Sequence
  3-E21-L2 Light Chain Variable Region
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYFYPLTFGQGTRLEIK

SEQ ID NO: 159 - protein, 119 aa, Artificial Sequence
  3-P21-H1 Heavy Chain Variable Region
EIQLVESGGGLVQPGGSLRLSCAASGYSFTGYNIHWVRQAPGKGLEWIGYINPYFGSTDYADSVKGRATLSVDKSKNTAYLQMNSLRAEDTAVYYCARGAYYGNAMDYWGQGTLVTVSS

SEQ ID NO: 160 - protein, 119 aa, Artificial Sequence
  3-P21-H2 Heavy Chain Variable Region
EIQLVESGGGLVQPGGSLRLSCAASGYSFTGYNMKWVRQAPGKGLEWIGNINPYFGSTNYADSVKGRATLSVDKSKNTAYLQMNSLRAEDTAVYYCARGAYYGNAMDYWGQGTLVTVSS

SEQ ID NO: 161 - protein, 119 aa, Artificial Sequence
  3-P21-H3 Heavy Chain Variable Region
EIQLVQSGAEVKKPGESLKISCKASGYSFTGYNIGWVRQMPGKGLEWIGIINPYFGSTRYSPSFQGQATLSVDKSISTAYLQWSSLKASDTAMYYCARGAYYGNAMDYWGQGTLVTVSS

SEQ ID NO: 162 - protein, 119 aa, Artificial Sequence
  3-P21-H4 Heavy Chain Variable Region
EIQLVQSGAEVKKPGESLKISCKASGYSFTGYNMKWVRQMPGKGLEWIGIINPYFGSTNYSPSFQGQATLSVDKSISTAYLQWSSLKASDTAMYYCARGAYYGNAMDYWGQGTLVTVSS

SEQ ID NO: 163 - protein, 113 aa, Artificial Sequence
  3-P21-L1 Light Chain Variable Region
DIQMTQSPSSLSASVGDRVTITCRSSQSLLNSGNQKNYVTWYQQKPGKAPKLLIYWASFLYSGVPSRFSGSGSGTDFTLTISSLQPEDFATYYCQNDYFYPLTFGQGTKVEIK

SEQ ID NO: 164 - protein, 113 aa, Artificial Sequence
  3-P21-L2 Light Chain Variable Region
DIQMTQSPSSLSASVGDRVTITCRSSQSLLNSGNQKNYVTWYQQKPGKAPKLLIYWASTRESGVPSRFSGSGSGTDFTLTISSLQPEDFATYYCQNDYFYPLTFGQGTKVEIK

SEQ ID NO: 165 - protein, 113 aa, Artificial Sequence
  3-P21-L3 Light Chain Variable Region
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYFYPLTFGQGTKVEIK

SEQ ID NO: 166 - protein, 119 aa, Artificial Sequence
  8-G12-H1 Heavy Chain Variable Region
EVQLVESGGGLVQPGGSLRLSCAASGYAFSDYWMNWVRQAPGKGLEWIGQIYPGYGDTKHNQRFMDRATLSADKSTSTAYMQMNSLRAEDTAVYFCARWGYYGNAMDYWGQGTLVTVSS

SEQ ID NO: 167 - protein, 119 aa, Artificial Sequence
  8-G12-H2 Heavy Chain Variable Region
QVQLVQSGAEVKKPGASVKVSCKASGYAFSDYWMNWVRQAPGQGLEWIGQIYPGYGDTKYAQKFQGRATLTADKSISTAYMELSRLRSDDTAVYFCARWGYYGNAMDYWGQGTLVTVSS

SEQ ID NO: 168 - protein, 113 aa, Artificial Sequence
  8-G12-L1 Light Chain Variable Region
DIQMTQSPSSLSASVGDRVTITCKSSQSLLNSGNQKNYLTWYQQKPGKAPKLLIYWASTRESGVPSRFSGSGSGTDFTLTISSLQPEDFATYYCQNAYIYPLTFGQGTKVEIK

SEQ ID NO: 169 - protein, 113 aa, Artificial Sequence
  8-G12-L2 Light Chain Variable Region
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNAYIYPLTFGGGTKVEIK

SEQ ID NO: 170 - protein, 118 aa, Artificial Sequence
  10-K2-H1 Heavy Chain Variable Region
EVQLVESGGGLVQPGGSLRLSCAASGYAFTSYVMHWVRQAPGKGLEWIGYINPYSDGTRHNQRFMDRATLSSDKSTSTAYMQMNSLRAEDTAVYYCTRIYYGNAMDYWGQGTLVTVSS

SEQ ID NO: 171 - protein, 118 aa, Artificial Sequence
  10-K2-H2 Heavy Chain Variable Region
QVQLVQSGAEVRKPGASVTVSCKASGYAFTSYVMHWVRQAPGQGLEWIGYINPYSDGTRFAQKFKGRATLTSDKSTSTAFMELSSLRSDDTAIYYCTRIYYGNAMDYWGQGTLVTVSS

SEQ ID NO: 172 - protein, 118 aa, Artificial Sequence
  10-K2-H3 Heavy Chain Variable Region
QVQLVQSGAEVKKPGASVKVSCKASGYAFTSYVMHWVRQAPGQGLEWIGYINPYSDGTRFAQKFKGRVTLTSDKSTSTAYMELSSLRSDDTAVYYCTRIYYGNAMDYWGQGTLVTVSS

SEQ ID NO: 173 - protein, 113 aa, Artificial Sequence
  10-K2-L1 Light Chain Variable Region
DIQMTQSPSSLSASVGDRVTITCKSSQSLLNSGNQKNYLTWYQQKPGKAPKLLIYWASTRESGVPSRFSGSGSGTDFTLTISSLQPEDFATYYCQNDYSYPFTFGQGTKVEIK

SEQ ID NO: 174 - protein, 113 aa, Artificial Sequence
  10-K2-L2 Light Chain Variable Region
DIVMTQSPLSLPVTPGEAASISCKSSQSLLNSGNQKNYLTWYLQKPGQSPQLLIYWASTRESGVPHRFSGSGSGTEFTLKISRVEAEDVGVYYCQNDYSYPFTFGQGTKVEIK

SEQ ID NO: 175 - protein, 118 aa, Artificial Sequence
  15-D6-H1 Heavy Chain Variable Region
EVQLVESGGGLVQPGGSLRLSCAASGYTFTSYWINWVRQAPGKGLEWIGDIYPGRSSTNYNQNFKDRATLSVDTSKNTAYLQMNSLRAEDTAVYYCSRLSRGNAMDYWGQGTLVTVSS

SEQ ID NO: 176 - protein, 118 aa, Artificial Sequence
  15-D6-H2 Heavy Chain Variable Region
EVQLVESGGGLVQPGGSLRLSCAASGYTFTSYWINWVRQAPGKGLEWIGDIYPGRSSTNYNQNFKGRATLSVDTSKNTAYLQMNSLRAEDTAVYYCSRLSRGNAMDYWGQGTLVTVSS

SEQ ID NO: 177 - protein, 118 aa, Artificial Sequence
  15-D6-H3 Heavy Chain Variable Region
EVQLVESGGGLVQPGGSLRLSCAASGYTFTSYWINWVRQAPGKGLEWIGNIYPGRSSTNYNQNFKGRATLSVDTSKNTAYLQMNSLRAEDTAVYYCSRLSRGNAMDYWGQGTLVTVSS

SEQ ID NO: 178 - protein, 118 aa, Artificial Sequence
  15-D6-H4 Heavy Chain Variable Region
EVQLVESGGGLVQPGGSLRLSCAASGYTFTSYWIHWVRQAPGKGLEWIGYIYPGRSSTNYNEKFKGRATLSVDTSKNTAYLQMNSLRAEDTAVYYCSRLSRGNAMDYWGQGTLVTVSS

SEQ ID NO: 179 - protein, 118 aa, Artificial Sequence
  15-D6-H5 Heavy Chain Variable Region
EVQLVESGGGLVQPGGSLRLSCAASGYTFTSYWINWVRQAPGKGLEWIGYIYPGRSSTNYNEKFKGRATLSVDTSKNTAYLQMNSLRAEDTAVYYCSRLSRGNAMDYWGQGTLVTVSS

SEQ ID NO: 180 - protein, 118 aa, Artificial Sequence
  15-D6-H6 Heavy Chain Variable Region
EVQLVESGGGLVQPGGSLRLSCAASGYTFTSYWINWVRQAPGKGLEWIGNIYPGRSSTNYNEKFKGRATLSVDTSKNTAYLQMNSLRAEDTAVYYCSRLSRGNAMDYWGQGTLVTVSS

SEQ ID NO: 181 - protein, 113 aa, Artificial Sequence
  15-D6-L1 Light Chain Variable Region
DIQMTQSPSSLSASVGDRVTITCRSSQSLLNSGNQKSYMTWYQQKPGKAPKLLIYWASNHASGVPSRFSGSGSGTDFTLTISSLQPEDFATYYCQNDYYYPFTFGQGTKVEIK

SEQ ID NO: 182 - protein, 113 aa, Artificial Sequence
  15-D6-L2 Light Chain Variable Region
DIQMTQSPSSLSASVGDRVTITCRSSQSLLNSGNQKSYMTWYQQKPGKAPKLLIYWASTRESGVPSRFSGSGSGTDFTLTISSLQPEDFATYYCQNDYYYPFTFGQGTKVEIK

SEQ ID NO: 183 - protein, 113 aa, Artificial Sequence
  15-D6-L3 Light Chain Variable Region
DIQMTQSPSSLSASVGDRVTITCRSSQSLLNSGNQKSYLTWYQQKPGKAPKLLIYWASNHASGVPSRFSGSGSGTDFTLTISSLQPEDFATYYCQNDYYYPFTFGQGTKVEIK

SEQ ID NO: 184 - protein, 113 aa, Artificial Sequence
  15-D6-L4 Light Chain Variable Region
DIQMTQSPSSLSASVGDRVTITCRSSQSLLNSGNQKSYVTWYQQKPGKAPKLLIYWASHRYTGVPSRFSGSGSGTDFTLTISSLQPEDFATYYCQNDYYYPFTFGQGTKVEIK

SEQ ID NO: 185 - protein, 113 aa, Artificial Sequence
  15-D6-L5 Light Chain Variable Region
DIQMTQSPSSLSASVGDRVTITCRSSQSLLNSGNQKSYVTWYQQKPGKAPKLLIYWASHRYTGVPSRFSGSGSGTEFTLTISSLQPEDFATYYCQNDYYYPFTFGQGTKVEIK

SEQ ID NO: 186 - protein, 118 aa, Artificial Sequence
  10-J10-H1 Heavy Chain Variable Region
QVQLVQSGAEVKKPGSSVKVSCKASGYTFTRYRISWVRQAPGQGLEWIGGIDPSDSETNYAQKFQGRATLTVDKSTSTAYMELSSLRSEDTAVYYCARLNYGNCFDYWGQGTLVTVSS

SEQ ID NO: 187 - protein, 118 aa, Artificial Sequence
  10-J10-H2 Heavy Chain Variable Region
QVQLVQSGAEVKKPGSSVKVSCKASGYTFTRYRISWVRQAPGQGLEWIGGIDPSDSETNYAQKFQGRATLTADKSTSTAYMELSSLRSEDTAVYYCARLNYGNCFDYWGQGTLVTVSS

SEQ ID NO: 188 - protein, 113 aa, Artificial Sequence
  10-J10-L1 Light Chain Variable Region
DIVMTQSPDSLAVSLGERATINCKSSQTLLNSGNQKNYLTWYQQKPGQPPKLLIYWASTRESGVPDRFSGSGSGTDFTLTISSLQAEDVAVYYCQNDYFYPFTFGQGTRLEIK

SEQ ID NO: 189 - protein, 113 aa, Artificial Sequence
  10-J10-L2 Light Chain Variable Region
DIVMTQSPDSLAVSLGERATINCKSSQTLLNSGNQKNYLAWYQQKPGQPPKLLIYWASTRESGVPDRFSGSGSGTDFTLTISSLQAEDVAVYYCQNDYFYPFTFGQGTKVEIK

SEQ ID NO: 190 - protein, 113 aa, Artificial Sequence
  10-J10-L3 Light Chain Variable Region
DIVMTQSPDSLAVSLGERATINCKSSQTLLNSGNQKNYLTWYQQKPGQPPKLLIYWASTRESGVPDRFSGSGSGTDFTLTISSLQAEDVAVYYCQNDYFYPFTFGQGTKVEIK

SEQ ID NO: 191 - protein, 117 aa, Artificial Sequence
  2-P8-H1 Heavy Chain Variable Region
QVQLVQSGAEVKKPGSSVKVSCKASGYSFTGYNLHWVRQAPGQGLEWIGWIDPYNGVTQYNEKFKGRATLTVDKSTSTAYMELSSLRSEDTAVYYCARWGGNYVDYWGQGTTVTVSS

SEQ ID NO: 192 - protein, 117 aa, Artificial Sequence
  2-P8-H2 Heavy Chain Variable Region
QVQLVQSGAEVKKPGASVKVSCKASGYSFTGYNLHWVRQAPGQGLEWIGWIDPYNGVTQYNEKFKGRVTITVDKSTSTAYMELSSLRSEDTAVYYCARWGGNYVDYWGQGTTVTVSS

SEQ ID NO: 193 - protein, 117 aa, Artificial Sequence
  2-P8-H3 Heavy Chain Variable Region
QVQLVQSGAEVKKPGSSVKVSCKASGYSFTGYNINWVRQAPGQGLEWIGWIDPYNGVTKYNEKFKGRATLTVDKSTNTAYMELSSLRSEDTAFYYCARWGGNYVDYWGQGTLVTVSS

SEQ ID NO: 194 - protein, 107 aa, Artificial Sequence
  2-P8-L1 Light Chain Variable Region
DIQMTQSPSSLSASVGDRVTITCRASQDINRYVSWFQQKPGKAPKTLIYRANYRYSGVPSRFSGSFSGQDYTLTISSLQPEDFATYYCLQYDEFPLTFGQGTKVEIK

SEQ ID NO: 195 - protein, 107 aa, Artificial Sequence
  2-P8-L2 Light Chain Variable Region
DIQMTQSPSSLSASVGDRVTITCKASQDINRYVSWFQQKPGKAPKTLIYRANYRYSGVPSRFSGSFSGQDYTLTISSLQPEDFATYYCLQYDEFPLTFGQGTKVEIK

SEQ ID NO: 196 - protein, 107 aa, Artificial Sequence
  2-P8-L3 Light Chain Variable Region
DIQMTQSPSSLSASVGDRVTITCKASQDINRYVSWFQQKPGKAPKSLIYRANYRYSGVPSRFSGSGSGQDYTLTISSLQPEDFATYYCLQYDEFPLTFGQGTKVEIK

SEQ ID NO: 197 - protein, 107 aa, Artificial Sequence
  2-P8-L4 Light Chain Variable Region
DIQMTQSPSTLSASVGDRVTITCRASQDINRYLSWFQQKPGKAPKTLIYRANNLASGVPSRFSGSFSGQEYTLTISSLQPDDFATYYCLQYDEFPLTFGQGTKVEIK

SEQ ID NO: 198 - protein, 246 aa, Artificial Sequence
  2-C3-H2(G4S)3L2
QVTLRESGPALVKPTQTLTLTCTFSGFTFSSYGMSWIRQPPGKALEWLATISGGGSYTYYLDSLKDRFTISRDISKNQVVLTVTNMDPADTATYFCARQSRGNAMDYWGQGTTVTVSSGGGGSGGGGSGGGGSDIQMTQSPSTLSASVGDRVTITCKSSQSLLNSGNQKNYLTWYQQKPGKAPKLLIYWASTRESGVPSRFSGSGSGTEFTLTISSLQPDDFATYYCQNDYSYPLTFGGGTKVEIK

SEQ ID NO: 199 - protein, 251 aa, Artificial Sequence
  2-C3-H2(G4S)4L2
QVTLRESGPALVKPTQTLTLTCTFSGFTFSSYGMSWIRQPPGKALEWLATISGGGSYTYYLDSLKDRFTISRDISKNQVVLTVTNMDPADTATYFCARQSRGNAMDYWGQGTTVTVSSGGGGSGGGGSGGGGSGGGGSDIQMTQSPSTLSASVGDRVTITCKSSQSLLNSGNQKNYLTWYQQKPGKAPKLLIYWASTRESGVPSRFSGSGSGTEFTLTISSLQPDDFATYYCQNDYSYPLTFGGGTKVEIK

SEQ ID NO: 200 - protein, 246 aa, Artificial Sequence
  2-C3-L2(G4S)3H2
DIQMTQSPSTLSASVGDRVTITCKSSQSLLNSGNQKNYLTWYQQKPGKAPKLLIYWASTRESGVPSRFSGSGSGTEFTLTISSLQPDDFATYYCQNDYSYPLTFGGGTKVEIKGGGGSGGGGSGGGGSQVTLRESGPALVKPTQTLTLTCTFSGFTFSSYGMSWIRQPPGKALEWLATISGGGSYTYYLDSLKDRFTISRDISKNQVVLTVTNMDPADTATYFCARQSRGNAMDYWGQGTTVTVSS

SEQ ID NO: 201 - protein, 247 aa, Artificial Sequence
  6-J11-H1(G4S)3L1
EVQLVESGGGLVQPGGSLRLSCAASGFIFSSFGMHWVRQAPGKGLEWVAYISSGRSTMYYADSVKGRFTISRDNSKNTLYLQMNSLTAEDTAVYYCARGGFYGNSLDYWGQGTLVTVSSGGGGSGGGGSGGGGSDIQMTQSPSSLSASVGDRVTITCKSSLSLLNSGNQKNYLTWYQQKPGKAPKLLIYWASTRESGVPSRFSGSGSGTDFTLTISSLQPEDFATYSCQNAYSYPLTFGQGTKVEIK

SEQ ID NO: 202 - protein, 252 aa, Artificial Sequence
  6-J11-H1(G4S)4L1
EVQLVESGGGLVQPGGSLRLSCAASGFIFSSFGMHWVRQAPGKGLEWVAYISSGRSTMYYADSVKGRFTISRDNSKNTLYLQMNSLTAEDTAVYYCARGGFYGNSLDYWGQGTLVTVSSGGGGSGGGGSGGGGSGGGGSDIQMTQSPSSLSASVGDRVTITCKSSLSLLNSGNQKNYLTWYQQKPGKAPKLLIYWASTRESGVPSRFSGSGSGTDFTLTISSLQPEDFATYSCQNAYSYPLTFGQGTKVEIK

SEQ ID NO: 203 - protein, 247 aa, Artificial Sequence
  6-J11-L1(G4S)3H1
DIQMTQSPSSLSASVGDRVTITCKSSLSLLNSGNQKNYLTWYQQKPGKAPKLLIYWASTRESGVPSRFSGSGSGTDFTLTISSLQPEDFATYSCQNAYSYPLTFGQGTKVEIKGGGGSGGGGSGGGGSEVQLVESGGGLVQPGGSLRLSCAASGFIFSSFGMHWVRQAPGKGLEWVAYISSGRSTMYYADSVKGRFTISRDNSKNTLYLQMNSLTAEDTAVYYCARGGFYGNSLDYWGQGTLVTVSS

SEQ ID NO: 204 - protein, 240 aa, Artificial Sequence
  5-E22-H3(G4S)3L3
QVQLVQSGAEVKKPGASVKVSCKASGYTITDNYMHWVRQAPGQGLEWIGEIYPGSGNTYYAEKFKNRATLTADKSISTAYMELSRLRSDDTAVYFCARGFPYYAMDYWGQGTLVTVSSGGGGSGGGGSGGGGSDIVMTQSPLSLPVTPGEPASISCHARQNINVWLSWYLQKPGQSPQLLIYKASNLHTGVPDRFSGSGSGTDFTLKISRVEAEDVGVYYCQQGQNYPLTFGQGTKVEIK

SEQ ID NO: 205 - protein, 245 aa, Artificial Sequence
  5-E22-H3(G4S)4L3
QVQLVQSGAEVKKPGASVKVSCKASGYTITDNYMHWVRQAPGQGLEWIGEIYPGSGNTYYAEKFKNRATLTADKSISTAYMELSRLRSDDTAVYFCARGFPYYAMDYWGQGTLVTVSSGGGGSGGGGSGGGGSGGGGSDIVMTQSPLSLPVTPGEPASISCHARQNINVWLSWYLQKPGQSPQLLIYKASNLHTGVPDRFSGSGSGTDFTLKISRVEAEDVGVYYCQQGQNYPLTFGQGTKVEIK

SEQ ID NO: 206 - protein, 240 aa, Artificial Sequence
  5-E22-L3(G4S)3H3
DIVMTQSPLSLPVTPGEPASISCHARQNINVWLSWYLQKPGQSPQLLIYKASNLHTGVPDRFSGSGSGTDFTLKISRVEAEDVGVYYCQQGQNYPLTFGQGTKVEIKGGGGSGGGGSGGGGSQVQLVQSGAEVKKPGASVKVSCKASGYTITDNYMHWVRQAPGQGLEWIGEIYPGSGNTYYAEKFKNRATLTADKSISTAYMELSRLRSDDTAVYFCARGFPYYAMDYWGQGTLVTVSS

SEQ ID NO: 207 - protein, 246 aa, Artificial Sequence
  3-E21-H3(G4S)3L2
EVQLLESGGGLVQPGGSLRLSCAASGFTFSKYAMSWVRQAPGKGLEWVAAISNGGSYTYYADSVKGRFTISRDNSKNTLYLQMNSLRAEDTAVYYCARHDKGNALDYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYFYPLTFGQGTRLEIK

SEQ ID NO: 208 - protein, 251 aa, Artificial Sequence
  3-E21-H3(G4S)4L2
EVQLLESGGGLVQPGGSLRLSCAASGFTFSKYAMSWVRQAPGKGLEWVAAISNGGSYTYYADSVKGRFTISRDNSKNTLYLQMNSLRAEDTAVYYCARHDKGNALDYWGQGTLVTVSSGGGGSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYFYPLTFGQGTRLEIK

SEQ ID NO: 209 - protein, 246 aa, Artificial Sequence
  3-E21-L2(G4S)3H3
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYFYPLTFGQGTRLEIKGGGGSGGGGSGGGGSEVQLLESGGGLVQPGGSLRLSCAASGFTFSKYAMSWVRQAPGKGLEWVAAISNGGSYTYYADSVKGRFTISRDNSKNTLYLQMNSLRAEDTAVYYCARHDKGNALDYWGQGTLVTVSS

SEQ ID NO: 210 - protein, 247 aa, Artificial Sequence
  3-P21-H2(G4S)3L1
EIQLVESGGGLVQPGGSLRLSCAASGYSFTGYNMKWVRQAPGKGLEWIGNINPYFGSTNYADSVKGRATLSVDKSKNTAYLQMNSLRAEDTAVYYCARGAYYGNAMDYWGQGTLVTVSSGGGGSGGGGSGGGGSDIQMTQSPSSLSASVGDRVTITCRSSQSLLNSGNQKNYVTWYQQKPGKAPKLLIYWASFLYSGVPSRFSGSGSGTDFTLTISSLQPEDFATYYCQNDYFYPLTFGQGTKVEIK

SEQ ID NO: 211 - protein, 252 aa, Artificial Sequence
  3-P21-H2(G4S)4L1
EIQLVESGGGLVQPGGSLRLSCAASGYSFTGYNMKWVRQAPGKGLEWIGNINPYFGSTNYADSVKGRATLSVDKSKNTAYLQMNSLRAEDTAVYYCARGAYYGNAMDYWGQGTLVTVSSGGGGSGGGGSGGGGSGGGGSDIQMTQSPSSLSASVGDRVTITCRSSQSLLNSGNQKNYVTWYQQKPGKAPKLLIYWASFLYSGVPSRFSGSGSGTDFTLTISSLQPEDFATYYCQNDYFYPLTFGQGTKVEIK

SEQ ID NO: 212 - protein, 247 aa, Artificial Sequence
  3-P21-L1(G4S)3H2
DIQMTQSPSSLSASVGDRVTITCRSSQSLLNSGNQKNYVTWYQQKPGKAPKLLIYWASFLYSGVPSRFSGSGSGTDFTLTISSLQPEDFATYYCQNDYFYPLTFGQGTKVEIKGGGGSGGGGSGGGGSEIQLVESGGGLVQPGGSLRLSCAASGYSFTGYNMKWVRQAPGKGLEWIGNINPYFGSTNYADSVKGRATLSVDKSKNTAYLQMNSLRAEDTAVYYCARGAYYGNAMDYWGQGTLVTVSS

SEQ ID NO: 213 - protein, 246 aa, Artificial Sequence
  15-D6-H5(G4S)3L4
EVQLVESGGGLVQPGGSLRLSCAASGYTFTSYWINWVRQAPGKGLEWIGYIYPGRSSTNYNEKFKGRATLSVDTSKNTAYLQMNSLRAEDTAVYYCSRLSRGNAMDYWGQGTLVTVSSGGGGSGGGGSGGGGSDIQMTQSPSSLSASVGDRVTITCRSSQSLLNSGNQKSYVTWYQQKPGKAPKLLIYWASHRYTGVPSRFSGSGSGTDFTLTISSLQPEDFATYYCQNDYYYPFTFGQGTKVEIK

SEQ ID NO: 214 - protein, 251 aa, Artificial Sequence
  15-D6-H5(G4S)4L4
EVQLVESGGGLVQPGGSLRLSCAASGYTFTSYWINWVRQAPGKGLEWIGYIYPGRSSTNYNEKFKGRATLSVDTSKNTAYLQMNSLRAEDTAVYYCSRLSRGNAMDYWGQGTLVTVSSGGGGSGGGGSGGGGSGGGGSDIQMTQSPSSLSASVGDRVTITCRSSQSLLNSGNQKSYVTWYQQKPGKAPKLLIYWASHRYTGVPSRFSGSGSGTDFTLTISSLQPEDFATYYCQNDYYYPFTFGQGTKVEIK

SEQ ID NO: 215 - protein, 246 aa, Artificial Sequence
  15-D6-L4(G4S)3H5
DIQMTQSPSSLSASVGDRVTITCRSSQSLLNSGNQKSYVTWYQQKPGKAPKLLIYWASHRYTGVPSRFSGSGSGTDFTLTISSLQPEDFATYYCQNDYYYPFTFGQGTKVEIKGGGGSGGGGSGGGGSEVQLVESGGGLVQPGGSLRLSCAASGYTFTSYWINWVRQAPGKGLEWIGYIYPGRSSTNYNEKFKGRATLSVDTSKNTAYLQMNSLRAEDTAVYYCSRLSRGNAMDYWGQGTLVTVSS

(19) United States
(12) Patent Application Publication
(10) Pub. No.: US 2022/0184126 A1
(43) Pub. Date: Jun. 16, 2022

(54) HUMANIZED ANTI-CLAUDIN 18.2 CHIMERIC ANTIGEN RECEPTORS AND USES THEREOF

(71) Applicant: Phanes Therapeutis, Inc., San Diego, CA (US)

(72) Inventors: Minghan Wang, San Diego, CA (US); Hui Zou, San Diego, CA (US); Haiqun Jia, San Diego, CA (US)

(21) Appl. No.: 17/593,066

(22) PCT Filed: Mar. 24, 2020
(86) PCT No.: PCT/US2020/024432

§ 371 Date: Sep. 8, 2021

Related U.S. Application Data
(60) Provisional application No. 62/825,955, filed on Mar. 29, 2019.
(60) Provisional application No. 62/859,843, filed on Jun. 11, 2019.
(60) Provisional application No. 62/896,758, filed on Sep. 6, 2019.

(51) Int. Cl. A61K 35/17; C07K 16/28; C07K 14/725; C12N 5/0783; A61P 35/00

(52) U.S. Cl. CPC A61K 35/17; C07K 16/28; A61P 35/00; C12N 5/0636; C07K 14/7051

(57) ABSTRACT

Chimeric antigen receptors (CARs) specific to CLDN18.2, vectors encoding the CLDN18.2 CAR, recombinant host cells comprising the CLDN18.2 CAR (CAR-Ts or CAR-NKs), and methods of using the CAR-Ts or CAR-NKs to treat a disease associated with the expression of CLDN18.2 thereof are described.

### CROSS REFERENCE TO RELATED APPLICATIONS

This application claims priority to U.S. Provisional Application No. 62/896,758, filed on Sep. 6, 2019; U.S. Provisional Application No. 62/859,843, filed on Jun. 11, 2019; and U.S. Provisional Application No. 62/825,955, filed on Mar. 29, 2019. Each disclosure is incorporated herein by reference in its entirety.

### FIELD OF THE INVENTION

This invention relates to humanized anti-claudin18.2 (CLDN18.2) chimeric antigen receptors (CARs), nucleic acids and expression vectors encoding the CARs, T cells engineered to express the CARs (CAR-T) and NK cells engineered to express the CARs (CAR-NK). Methods of making the CARs, methods of making the CAR-Ts/CAR-NKs, and methods of using the CAR-Ts/CAR-NKs to treat a disease associated with the expression of CLDN18.2, including cancer, are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065799.19WO1 Sequence Listing” and a creation date of Mar. 11, 2020 and having a size of 147 kb. The sequence listing submitted via EFS-Web is part of the specification and is herein incorporated by reference in its entirety.

### BACKGROUND OF THE INVENTION

The standard of care anti-cancer medicines provides significant benefits. Recently, the availability of immuno-oncology drugs, such as anti-PD-1 mAbs, anti-PD-L1 mAbs and anti-CD3 bispecific T-cell engagers, has advanced the concept of leveraging and activating patients' immune system to fight various types of cancer. However, poor response, insufficient efficacy, and/or safety issues remain to be resolved. CAR-T (chimeric antigen receptor-T) cell therapies involve genetically engineering a patient's own immune cells, such as T cells, and redirecting them to a suitable cell surface antigen on cancer cells (Mayor et al., Immunotherapy. 2016; 8:491-494). This approach has demonstrated success in patients suffering from chemorefractory B cell malignancies and other cancers (Pettitt et al., Mol Ther. 2018; 26:342-353). T cells can be engineered to possess specificity to one or more cancer cell surface targets/antigens to recognize and kill the cancer cell. The process includes transducing T cells with DNA or other genetic material encoding the chimeric antigen receptor (CAR), which comprises an extracellular antigen specific binding domain, such as one or more single chain variable fragments (scFv) of a monoclonal antibody, a hinge and transmembrane region, and an intracellular signaling domain (including one or more costimulatory domains and one or more activating domains) (Kochenderfer et al., Nat Rev Clin Oncol. 2013; 10:267-276). CAR-expressing immune cells, such as T cells and NK cells, can be used to treat various diseases, including liquid and solid tumors. Successful CAR-T cell therapies can specifically recognize and destroy targeted cells and maintain the ability to persist and proliferate over time.

Claudin 18.2 (CLDN18.2), also known as claudin-18a2.1, is a member of the claudin (CLDN) family transmembrane proteins of at least 27 isoforms in humans. Claudins are the major structural components of tight junction between epithelial cells and function as ion pores to regulate the paracellular permeability of cations and anions (Sahin et al., Physiol Rev. 2013; 93:525-569). The expression of CLDN18 is normally limited to lung and stomach tissues. CLDN18 has two splicing variants. CLDN18.1 is the lung-specific variant whereas CLDN18.2 is the stomach-specific variant. The splicing variants differ at their N-terminal 69 amino acid residues due to alternative splicing of the first exon (Niimi et al., Mol Cell Biol. 2001; 21:7380-7390). Studies with CLDN18.2 knockout mice suggest that CLDN18.2 plays a critical role in preventing gastric acid leakage into the stomach lumen (Hayash et al., Gastroenterology 2012; 142:292-304).

Dysregulated expression of claudins are detected in many cancers and may contribute to tumorigenesis and cancer invasiveness (Singh et al., J Oncology 2010; 2010: 541957). The expression of CLDN18.2 is elevated in pancreatic ductal adenocarcinomas (PDAC) (Tanaka et al., J Histochem Cytochem. 2011; 59:942-952), esophageal tumors, non-small cell lung cancers (NSCLC), ovarian cancers (Sahin et al., Clin Cancer Res. 2008; 14:7624-7634), bile duct adenocarcinomas (Keira et al., Virchows Arch. 2015; 466:265-277), and cholangiocarcinomas (Shinozaki et al., Virchows Arch. 2011; 459:73-80). CLDN18.2 is an ideal target for CAR-T cell therapies to treat and cure CLDN18.2-positive cancers.

### BRIEF SUMMARY OF THE INVENTION

In one general aspect, the invention relates to a chimeric antigen receptor (CAR) construct that induces T cell mediated cancer killing, wherein the CAR construct comprises at least one antigen binding domain that specifically binds human claudin 18.2 (CLDN18.2), a hinge region, a transmembrane region, and an intracellular signaling domain.

Provided are isolated polynucleotides comprising a nucleic acid sequence encoding a chimeric antigen receptor (CAR). The CAR can comprise (a) an extracellular domain comprising at least one antigen binding domain that specifically binds claudin 18.2 (CLDN18.2); (b) a hinge region; (c) a transmembrane region; and (d) an intracellular signaling domain.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 21, 22, 23, 51, 52 and 53, respectively;
(2) SEQ ID NOs: 24, 25, 26, 54, 55 and 56, respectively;
(3) SEQ ID NOs: 27, 28, 29, 57, 58 and 59, respectively;
(4) SEQ ID NOs: 30, 31, 32, 60, 61 and 62, respectively;
(5) SEQ ID NOs: 33, 34, 35, 63, 64 and 65, respectively;
(6) SEQ ID NOs: 36, 37, 38, 66, 67 and 68, respectively;
(7) SEQ ID NOs: 39, 40, 41, 69, 70 and 71, respectively;
(8) SEQ ID NOs: 42, 43, 44, 72, 73 and 74, respectively;
(9) SEQ ID NOs: 45, 46, 47, 75, 76 and 77, respectively; or
(10) SEQ ID NOs: 48, 49, 50, 78, 79 and 80, respectively;
   wherein the antigen binding domain thereof specifically binds CLDN18.2, preferably human CLDN18.2.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 81, 82, 83, 111, 112 and 113, respectively;
(2) SEQ ID NOs: 84, 85, 86, 114, 115 and 116, respectively;
(3) SEQ ID NOs: 87, 88, 89, 117, 118 and 119, respectively;
(4) SEQ ID NOs: 90, 91, 92, 120, 121 and 122, respectively;
(5) SEQ ID NOs: 93, 94, 95, 123, 124 and 125, respectively;
(6) SEQ ID NOs: 96, 97, 98, 126, 127 and 128, respectively;
(7) SEQ ID NOs: 99, 100, 101, 129, 130 and 131, respectively;
(8) SEQ ID NOs: 102, 103, 104, 132, 133 and 134, respectively;
(9) SEQ ID NOs: 105, 106, 107, 135, 136 and 137, respectively; or
(10) SEQ ID NOs: 108, 109, 110, 138, 139 and 140, respectively;
   wherein the antigen binding domain thereof specifically binds CLDN18.2, preferably human CLDN18.2.

In certain embodiments, the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, 15, 17, or 19, or a light chain variable region having a polypeptide sequence at least 95%, at least 96%, at least 97%, at least 98%, or at least 99% identical to SEQ ID NO: 2, 4, 6, 8, 10, 12, 14, 16, 18, or 20.

In certain embodiments, the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18; or
(10) a heavy chain variable region having the polypeptide sequence of SEQ ID NO:19, and a light chain variable region having the polypeptide sequence of SEQ ID NO:20.

In certain embodiments, the antigen binding domain is humanized and comprises a heavy chain variable region having a polypeptide sequence at least 95%, at least 96%, at least 97%, at least 98%, or at least 99% identical to SEQ ID NO: 142, 143, 146, 147, 151, 152, 154, 155, 156, 159, 160, 161, 162, 166, 167, 170, 171, 172, 175, 176, 177, 178, 179, 180, 186, 187, 191, 192, or 193, or a light chain variable region having a polypeptide sequence at least 95%, at least 96%, at least 97%, at least 98%, or at least 99% identical to SEQ ID NO: 144, 145, 148, 149, 150, 153, 157, 158, 163, 164, 165, 168, 169, 173, 174, 181, 182, 183, 184, 185, 188, 189, 190, 194, 195, 196, or 197.

In certain embodiments, the antigen binding domain is humanized and comprises:
(1) a heavy chain variable region having the polypeptide sequence of SEQ ID NO:142, and a light chain variable region having the polypeptide sequence of SEQ ID NO:144;
(2) a heavy chain variable region having the polypeptide sequence of SEQ ID NO:142, and a light chain variable region having the polypeptide sequence of SEQ ID NO:145;
(3) a heavy chain variable region having the polypeptide sequence of SEQ ID NO:143, and a light chain variable region having the polypeptide sequence of SEQ ID NO:144;
(4) a heavy chain variable region having the polypeptide sequence of SEQ ID NO:143, and a light chain variable region having the polypeptide sequence of SEQ ID NO:145;
(5) a heavy chain variable region having the polypeptide sequence of SEQ ID NO:146, and a light chain variable region having the polypeptide sequence of SEQ ID NO: 48;
(6) a heavy chain variable region having the polypeptide sequence of SEQ ID NO:146, and a light chain variable region having the polypeptide sequence of SEQ ID NO: 149;
(7) a heavy chain variable region having the polypeptide sequence of SEQ ID NO:146, and a light chain variable region having the polypeptide sequence of SEQ ID NO: 50;
(8) a heavy chain variable region having the polypeptide sequence of SEQ ID NO:147, and a light chain variable region having the polypeptide sequence of SEQ ID NO: 48;
(9) a heavy chain variable region having the polypeptide sequence of SEQ ID NO:147, and a light chain variable region having the polypeptide sequence of SEQ ID NO:149;
(10) a heavy chain variable region having the polypeptide sequence of SEQ ID NO:147, and a light chain variable region having the polypeptide sequence of SEQ ID NO:150;
(11) a heavy chain variable region having the polypeptide sequence of SEQ ID NO:151, and a light chain variable region having the polypeptide sequence of SEQ ID NO:153;
(12) a heavy chain variable region having the polypeptide sequence of SEQ ID NO:152, and a light chain variable region having the polypeptide sequence of SEQ ID NO:153;
(13) a heavy chain variable region having the polypeptide sequence of SEQ ID NO:154, and a light chain variable region having the polypeptide sequence of SEQ ID NO:157;
(14) a heavy chain variable region having the polypeptide sequence of SEQ ID NO:155, and a light chain variable region having the polypeptide sequence of SEQ ID NO:157;
(15) a heavy chain variable region having the polypeptide sequence of SEQ ID NO:156, and a light chain variable region having the polypeptide sequence of SEQ ID NO:158;
(16) a heavy chain variable region having the polypeptide sequence of SEQ ID NO:159, and a light chain variable region having the polypeptide sequence of SEQ ID NO:163;
(17) a heavy chain variable region having the polypeptide sequence of SEQ ID NO:159, and a light chain variable region having the polypeptide sequence of SEQ ID NO:164;
(18) a heavy chain variable region having the polypeptide sequence of SEQ ID NO:160, and a light chain variable region having the polypeptide sequence of SEQ ID NO:163;
(19) a heavy chain variable region having the polypeptide sequence of SEQ ID NO:160, and a light chain variable region having the polypeptide sequence of SEQ ID NO:164;
(20) a heavy chain variable region having the polypeptide sequence of SEQ ID NO:161, and a light chain variable region having the polypeptide sequence of SEQ ID NO:165; or
(21) a heavy chain variable region having the polypeptide sequence of SEQ ID NO:162, and a light chain variable region having the polypeptide sequence of SEQ ID NO:165.

In certain embodiments, the antigen binding domain is a single chain variable fragment (scFv) that specifically binds CLDN18.2, preferably human CLDN18.2.

In certain embodiments, the antigen binding domain is a humanized single chain variable fragment (scFv) that specifically binds CLDN18.2, preferably human CLDN18.2. In certain embodiments, the humanized single chain variable fragment (scFv) comprises a polypeptide sequence at least 95% identical to any one of SEQ ID NOs: 198-215.

In certain embodiments, the chimeric antigen receptor (CAR) comprises one or more antigen binding domains.

In certain embodiments, the intracellular signaling domain comprises one or more costimulatory domains and one or more activating domains.

Also provided are chimeric antigen receptors (CARs) encoded by the isolated polynucleotides of the invention.

Also provided are vectors comprising the isolated polynucleotides comprising nucleic acids encoding the CARs of the invention.

Also provided are host cells comprising the vectors of the invention.

In certain embodiments, the host cell is a T cell, preferably a human T cell. In certain embodiments, the host cell is a NK cell, preferably a human NK cell. The T cell or NK cell can, for example, be engineered to express the CAR of the invention to treat diseases such as cancer.

Also provided are methods of making a host cell expressing a chimeric antigen receptor (CAR) of the invention. The methods comprise transducing a T cell or a NK cell with a vector comprising the isolated nucleic acids encoding the CARs of the invention.

Also provided are methods of producing a CAR-T cell or CAR-NK cell of the invention. The methods comprise culturing T cells or NK cells comprising the isolated polynucleotide comprising a nucleic acid encoding a chimeric antigen receptor (CAR) of the invention under conditions to produce the CAR-T cell or CAR-NK cell, and recovering the CAR-T cell or CAR-NK cell.

Also provided are methods of generating a population of RNA-engineered cells comprising a chimeric antigen receptor (CAR) of the invention. The methods comprise contacting a cell with the isolated polynucleotide comprising a nucleic acid encoding a chimeric antigen receptor (CAR) of the invention, wherein the isolated polynucleotide is an in vitro transcribed RNA or synthetic RNA.

Also provided are methods of treating cancer in a subject in need thereof, comprising administering to the subject the CAR-T cells and/or CAR-NK cells of the invention. The cancer can be any liquid or solid cancer, for example, it can be selected from, but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of treating an inflammatory disease in a subject in need thereof, comprising administering to the subject the CAR-T cells and/or CAR-NK cells of the invention.

In certain embodiments, the methods of treating cancer or inflammatory disease in a subject in need thereof further comprise administering to the subject in need thereof an agent that increases the efficacy of a cell expressing a CAR molecule.

In certain embodiments, the methods of treating cancer or inflammatory disease in a subject in need thereof further comprise administering to the subject in need thereof an agent that ameliorates one or more side effects associated with administration of a cell expressing a CAR molecule.

In certain embodiments, the methods of treating cancer or inflammatory disease in a subject in need thereof further comprise administering to the subject in need thereof an agent that treats the disease associated with Claudin 18.2.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B show the binding of humanized anti-CLDN18.2 mAbs to HEK293-CLDN18.2 and HEK293-CLDN18.1, which express the full-length human CLDN18.2 and CLDN18.1, respectively. The experiment was carried out by FACS analysis.

FIGS. 2A-2D show the binding of humanized anti-CLDN18.2 mAbs to HEK293-CLDN18.2 cells stably expressing full-length human CLDN18.2. The experiment was carried out by FACS analysis.

FIGS. 3A-3L show the binding of humanized scFvs to HEK293-CLDN18.2 cells stably expressing full-length human CLDN18.2. The experiment was carried out by FACS analysis.

FIG. 4 shows the tumor cell killing activity of the CART cells assembled with an anti-CLDN18.2 scFv against CLDN18.2-expressing cells (HEK293-CLDN18.2); CLDN18.1-expressing cells (HEK293-CLDN18.1) were used as control.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., chimeric antigen receptors (CARs) comprising antigen binding domains specific for CLDN18.2 and polynucleotides that encode them, CLDN18.2 polypeptides and CLDN18.2 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells, and are referred to as CAR-T cells because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention.

### Chimeric Antigen Receptor (CAR)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

In one aspect, the CAR comprises an antigen binding domain, a hinge region, a costimulatory domain, an activating domain and a transmembrane region. In one aspect, the CAR comprises an antigen binding domain, a hinge region, two costimulatory domains, an activating domain and a transmembrane region. In one aspect, the CAR comprises two antigen binding domains, a hinge region, a costimulatory domain, an activating domain and a transmembrane region. In one aspect, the CAR comprises two antigen binding domains, a hinge region, two costimulatory domains, an activating domain and a transmembrane region.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

### Costimulatory Domains

As used herein, chimeric antigen receptors can incorporate costimulatory (signaling) domains to increase their potency. A costimulatory (signaling) domain can be derived from a costimulatory molecule. Costimulatory molecules are cell surface molecules other than antigen receptors or their ligands that are required for an efficient immune response. Costimulatory domains can be derived from costimulatory molecules, which can include, but are not limited to CD28, CD28T, OX40, 4-1BB/CD137, CD2, CD3 (alpha, beta, delta, epsilon, gamma, zeta), CD4, CDS, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, or fragments or any combination thereof.

### Activating Domains

As used herein, chimeric antigen receptors can comprise activating domains. Activating domains can include, but are not limited to, CD3. CD3 is an element of the T cell receptor on native T cells and has been shown to be an important intracellular activating element in CARs. In a preferred embodiment, the CD3 is CD3 zeta.

### Hinge Region

As described herein, the chimeric antigen receptor can comprise a hinge region. This is a portion of the extracellular domain, sometimes referred to as a “spacer” region. A variety of hinges can be employed in accordance with the invention, including costimulatory molecules, as discussed above, immunoglobulin (Ig) sequences, or other suitable molecules to achieve the desired special distance from the target cell. In some embodiments, the entire extracellular region comprises a hinge region.

### Transmembrane Region

As used herein, chimeric antigen receptors (CARs) can comprise a transmembrane region/domain. The CAR can be designed to comprise a transmembrane domain that is fused to the extracellular domain of the CAR. It can similarly be fused to the intracellular domain of the CAR. In one embodiment, the transmembrane domain that is naturally associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention can be derived from (i.e. comprise or engineered from), but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, an immunoglobulin protein, or fragments or any combination thereof.

### Immune Cells

According to particular aspects, the invention provides cells that are immune cells that comprise the isolated polynucleotides or vectors comprising the isolated polynucleotides comprising the nucleotide sequence encoding the CAR are provided herein. The immune cells comprising the isolated polynucleotides and/or vectors of the invention can be referred to as “engineered immune cells.” Preferably, the engineered immune cells are derived from a human (are of human origin prior to being made recombinant).

The engineered immune cells can, for example, be cells of the lymphoid lineage. Non-limiting examples of cells of the lymphoid lineage can include T cells and Natural Killer (NK) cells. T cells express the T cell receptor (TCR), with most cells expressing α and β chains and a smaller population expressing γ and δ chains. T cells useful as engineered immune cells of the invention can be CD4⁺ or CD8⁺ and can include, but are not limited to, T helper cells (CD4⁺), cytotoxic T cells (also referred to as cytotoxic T lymphocytes, CTL; CD8⁺ cells), and memory T cells, including central memory T cells, stem-like memory T cells, and effector memory T cells, natural killer T cells, mucosal associated invariant T cells, and γδT cells. Other exemplary immune cells include, but are not limited to, macrophages, antigen presenting cells (APCs), or any immune cell that expresses an inhibitor of a cell-mediated immune response, for example, an immune checkpoint inhibitor pathway receptor (e.g., PD-1). Precursor cells of immune cells that can be used according to the invention, include, hematopoietic stem and/or progenitor cells. Hematopoietic stem and/or progenitor cells can be derived from bone marrow, umbilical cord blood, adult peripheral blood after cytokine mobilization, and the like, by methods known in the art. The immune cells are engineered to recombinantly express the CARs of the invention.

Immune cells and precursor cells thereof can be isolated by methods known in the art, including commercially available methods (see, e.g., Rowland Jones et al., Lymphocytes: A Practical Approach, Oxford University Press, NY (1999)). Sources for immune cells or precursors thereof include, but are not limited to, peripheral blood, umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolate or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells.

The immune cells or precursor cells thereof can be autologous or non-autologous to the subject to which they are administered in the methods of treatment of the invention. Autologous cells are isolated from the subject to which the engineered immune cells recombinantly expressing the CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

Various methods for isolating immune cells that can be used for recombinant expression of the CARs of the invention have been described previously, and can be used, including, but not limited to, using peripheral donor lymphocytes (Sadelain et al., Nat. Rev. Cancer 3:35-45 (2003); Morgan et al., Science 314:126-9 (2006)), using lymphocyte cultures derived from tumor infiltrating lymphocytes (TILs) in tumor biopsies (Panelli et al., J. Immunol. 164:495-504 (2000); Panelli et al., J. Immunol. 164:4382-92 (2000)) and using selectively in vitro expanded antigen-specific peripheral blood leukocytes employing artificial antigen-presenting cells (AAPCs) or dendritic cells (Dupont et al., Cancer Res. 65:5417-427 (2005); Papanicolaou et al., Blood 102:2498-505 (2003)). In the case of using stem cells, the cells can be isolated by methods well known in the art (see, e.g., Klug et al., Hematopoietic Stem Cell Protocols, Humana Press, NJ (2002); Freshney et al., Culture of Human Stem Cells, John Wiley & Sons (2007)).

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-g, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

### Antigen Binding Domain

As used herein, the term “antigen binding domain” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen binding domain is capable of binding to the same antigen to which the parent antibody binds. According to particular embodiments, the antigen binding domain comprises a single-chain antibody molecule (scFv).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term “single-chain antibody” refers to a conventional single-chain antibody in the field, which comprises a heavy chain variable region and a light chain variable region connected by a short peptide of about 5 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antigen binding domain” refers to a non-human antigen binding domain that is modified to increase the sequence homology to that of a human antibody, such that the antigen-binding properties of the antigen binding domain are retained, but its antigenicity in the human body is reduced.

As used herein, the term “chimeric antigen binding domain” refers to an antigen binding domain wherein the amino acid sequence of the immunoglobulin molecule is derived from two or more species. The variable region of both the light and heavy chains often corresponds to the variable region of an antigen binding domain derived from one species of mammal (e.g., mouse, rat, rabbit, etc.) having the desired specificity, affinity, and capability, while the constant regions correspond to the sequences of an antigen binding domain derived from another species of mammal (e.g., human) to avoid eliciting an immune response in that species.

As used herein, the term “CLDN18.2” refers to claudin 18 variant 2, claudin-18.2 or claudin-18a2.1, which belongs to the claudin family of transmembrane proteins. CLDN18.2 is specifically expressed on the surface of epithelial cells in stomach (Niimi et al., Mol Cell Biol.

2001; 21:7380-7390) and becomes one of the major structural components of the tight junction between the epithelial cells (Sahin et al., Physiol Rev. 2013; 93:525-569). The term “human CLDN18.2” refers to a CLDN18.2 originated from a human. An exemplary amino acid sequence of a human CLDN18.2 is represented in GenBank Accession No. AAL15637.1 (SEQ ID NO:141).

As used herein, an antigen binding domain that “specifically binds to CLDN18.2” refers to an antigen binding domain that binds to a CLDN18.2, preferably a human CLDN18.2, with a KD of 1×10⁻⁷ M or less, preferably 1×10⁻⁸M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antigen binding domains can be determined using methods in the art in view of the present disclosure. For example, the KD of an antigen binding domain can be determined by using surface plasmon resonance, such as by using a biosensor system, e.g., a Biacore® system, or by using bio-layer interferometry technology, such as an Octet RED96 system.

The smaller the value of the KD of an antigen binding domain, the higher affinity that the antigen binding domain binds to a target antigen.

According to a particular aspect, the invention relates to chimeric antigen receptors (CAR)s comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 21, 22, 23, 51, 52 and 53, respectively;
(2) SEQ ID NOs: 24, 25, 26, 54, 55 and 56, respectively;
(3) SEQ ID NOs: 27, 28, 29, 57, 58 and 59, respectively;
(4) SEQ ID NOs: 30, 31, 32, 60, 61 and 62, respectively;
(5) SEQ ID NOs: 33, 34, 35, 63, 64 and 65, respectively;
(6) SEQ ID NOs: 36, 37, 38, 66, 67 and 68, respectively;
(7) SEQ ID NOs: 39, 40, 41, 69, 70 and 71, respectively;
(8) SEQ ID NOs: 42, 43, 44, 72, 73 and 74, respectively;
(9) SEQ ID NOs: 45, 46, 47, 75, 76 and 77, respectively; or
(10) SEQ ID NOs: 48, 49, 50, 78, 79 and 80, respectively;
   wherein the antigen binding domain thereof specifically binds CLDN18.2, preferably human CLDN18.2.

According to another particular aspect, the invention relates to chimeric antigen receptors (CARs) comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 81, 82, 83, 111, 112 and 113, respectively;
(2) SEQ ID NOs: 84, 85, 86, 114, 115 and 116, respectively;
(3) SEQ ID NOs: 87, 88, 89, 117, 118 and 119, respectively;
(4) SEQ ID NOs: 90, 91, 92, 120, 121 and 122, respectively;
(5) SEQ ID NOs: 93, 94, 95, 123, 124 and 125, respectively;
(6) SEQ ID NOs: 96, 97, 98, 126, 127 and 128, respectively;
(7) SEQ ID NOs: 99, 100, 101, 129, 130 and 131, respectively;
(8) SEQ ID NOs: 102, 103, 104, 132, 133 and 134, respectively;
(9) SEQ ID NOs: 105, 106, 107, 135, 136 and 137, respectively; or
(10) SEQ ID NOs: 108, 109, 110, 138, 139 and 140, respectively;
   wherein the antigen binding domain thereof specifically binds CLDN18.2, preferably human CLDN18.2.

According to another particular aspect, the invention relates to an antigen binding domain comprising a heavy chain variable region having a polypeptide sequence at least 95%, at least 96%, at least 97%, at least 98%, or at least 99% identical to SEQ ID NO: 1, 3, 5, 7, 9, 11, 13, 15, 17, or 19, or a light chain variable region having a polypeptide sequence at least 95%, at least 96%, at least 97%, at least 98%, or at least 99% identical to SEQ ID NO: 2, 4, 6, 8, 10, 12, 14, 16, 18, or 20.

According to another particular aspect, the invention relates to an antigen binding domain, comprising:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18; or
(10) a heavy chain variable region having the polypeptide sequence of SEQ ID NO:19, and a light chain variable region having the polypeptide sequence of SEQ ID NO:20.

According to another particular aspect, the antigen binding domain is humanized and comprises a heavy chain variable region having a polypeptide sequence at least 95%, at least 96%, at least 97%, at least 98%, or at least 99% identical to SEQ ID NO: 142, 143, 146, 147, 151, 152, 154, 155, 156, 159, 160, 161, 162, 166, 167, 170, 171, 172, 175, 176, 177, 178, 179, 180, 186, 187, 191, 192, or 193, or a light chain variable region having a polypeptide sequence at least 95%, at least 96%, at least 97%, at least 98%, or at least 99% identical to SEQ ID NO: 144, 145, 148, 149, 150, 153, 157, 158, 163, 164, 165, 168, 169, 173, 174, 181, 182, 183, 184, 185, 188, 189, 190, 194, 195, 196, or 197.

According to another particular aspect, the antigen binding domain is humanized and comprises:
(1) a heavy chain variable region having the polypeptide sequence of SEQ ID NO:142, and a light chain variable region having the polypeptide sequence of SEQ ID NO: 44;
(2) a heavy chain variable region having the polypeptide sequence of SEQ ID NO:142, and a light chain variable region having the polypeptide sequence of SEQ ID NO: 45;
(3) a heavy chain variable region having the polypeptide sequence of SEQ ID NO:143, and a light chain variable region having the polypeptide sequence of SEQ ID NO: 44;
(4) a heavy chain variable region having the polypeptide sequence of SEQ ID NO:143, and a light chain variable region having the polypeptide sequence of SEQ ID NO: 145;
(5) a heavy chain variable region having the polypeptide sequence of SEQ ID NO:146, and a light chain variable region having the polypeptide sequence of SEQ ID NO: 48;
(6) a heavy chain variable region having the polypeptide sequence of SEQ ID NO:146, and a light chain variable region having the polypeptide sequence of SEQ ID NO: 49;
(7) a heavy chain variable region having the polypeptide sequence of SEQ ID NO:146, and a light chain variable region having the polypeptide sequence of SEQ ID NO:150;
(8) a heavy chain variable region having the polypeptide sequence of SEQ ID NO:147, and a light chain variable region having the polypeptide sequence of SEQ ID NO:148;
(9) a heavy chain variable region having the polypeptide sequence of SEQ ID NO:147, and a light chain variable region having the polypeptide sequence of SEQ ID NO:149;
(10) a heavy chain variable region having the polypeptide sequence of SEQ ID NO:147, and a light chain variable region having the polypeptide sequence of SEQ ID NO:150;
(11) a heavy chain variable region having the polypeptide sequence of SEQ ID NO:151, and a light chain variable region having the polypeptide sequence of SEQ ID NO:153;
(12) a heavy chain variable region having the polypeptide sequence of SEQ ID NO:152, and a light chain variable region having the polypeptide sequence of SEQ ID NO:153;
(13) a heavy chain variable region having the polypeptide sequence of SEQ ID NO:154, and a light chain variable region having the polypeptide sequence of SEQ ID NO:157;
(14) a heavy chain variable region having the polypeptide sequence of SEQ ID NO:155, and a light chain variable region having the polypeptide sequence of SEQ ID NO:157;
(15) a heavy chain variable region having the polypeptide sequence of SEQ ID NO:156, and a light chain variable region having the polypeptide sequence of SEQ ID NO:158;
(16) a heavy chain variable region having the polypeptide sequence of SEQ ID NO:159, and a light chain variable region having the polypeptide sequence of SEQ ID NO:163;
(17) a heavy chain variable region having the polypeptide sequence of SEQ ID NO:159, and a light chain variable region having the polypeptide sequence of SEQ ID NO:164;
(18) a heavy chain variable region having the polypeptide sequence of SEQ ID NO:160, and a light chain variable region having the polypeptide sequence of SEQ ID NO:163;
(19) a heavy chain variable region having the polypeptide sequence of SEQ ID NO:160, and a light chain variable region having the polypeptide sequence of SEQ ID NO:164;
(20) a heavy chain variable region having the polypeptide sequence of SEQ ID NO:161, and a light chain variable region having the polypeptide sequence of SEQ ID NO:165; or
(21) a heavy chain variable region having the polypeptide sequence of SEQ ID NO:162, and a light chain variable region having the polypeptide sequence of SEQ ID NO:165.

According to another particular aspect, the antigen binding domain is a single chain variable fragment (scFv) that specifically binds CLDN18.2, preferably human CLDN18.2.

In certain embodiments, the antigen binding domain is a humanized single chain variable fragment (scFv) that specifically binds CLDN18.2, preferably human CLDN18.2. In certain embodiments, the humanized single chain variable fragment (scFv) comprises a polypeptide sequence at least 95%, at least 96%, at least 97%, at least 98%, or at least 99% identical to any one of SEQ ID NOs:198-215. In certain embodiments, the humanized single chain variable fragment (scFv) comprises a polypeptide sequence having an amino acid sequence selected from the group consisting of SEQ ID NOs:198-215.

According to another particular aspect, the chimeric antigen receptor comprises one or more antigen binding domains.

According to another particular aspect, the intracellular signaling domain comprises one or more costimulatory domains and one or more activating domains.

In another general aspect, the invention relates to an isolated polynucleotide comprising a nucleic acid encoding a chimeric antigen receptor (CAR), wherein the CAR comprises an antigen binding domain thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antigen binding domains thereof of the invention can be altered without changing the amino acid sequences of the proteins.

In another general aspect, the invention relates to a vector comprising the isolated polynucleotide comprising the nucleic acid encoding the CAR, wherein the CAR comprises an antigen binding domain thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antigen binding domain thereof in the cell. Conventional cloning techniques or artificial gene synthesis can be used to generate a recombinant expression vector according to embodiments of the invention.

In another general aspect, the invention relates to a cell transduced with the vector comprising the isolated nucleic acids encoding the CARs of the invention. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny. In certain embodiments, the cell is a human CAR-T cell, wherein the T cell is engineered to express the CAR of the invention to treat diseases such as cancer. In certain embodiments, the cell is a human CAR-NK cell, wherein the NK cell engineered to express the CAR of the invention is used to treat diseases such as cancer.

In another general aspect, the invention relates to a method of making a CAR-T cell by transducing a T cell with a vector comprising the isolated nucleic acids encoding the CARs of the invention.

In another general aspect, the invention relates to a method of producing the CAR-T cell thereof of the invention, comprising culturing T-cells comprising a nucleic acid encoding a chimeric antigen receptor (CAR) of the invention under conditions to produce the CAR-T cell, and recovering the CAR-T cell.

In another general aspect, the invention relates to a method of making a CAR- NK cell by transducing a NK cell with a vector comprising the isolated nucleic acids encoding the CARs of the invention.

In another general aspect, the invention relates to a method of producing a CAR-NK cell of the invention, comprising culturing NK cells comprising nucleic acids encoding the chimeric antigen receptor (CAR) thereof under conditions to produce the CAR-NK cell, and recovering the CAR-NK cell.

In another general aspect, the invention relates to a method of generating a population of RNA-engineered cells comprising a chimeric antigen receptor (CAR) of the invention. The methods comprise contacting a population of cells with isolated polynucleotides comprising a nucleic acid encoding a CAR of the invention, wherein the isolated polynucleotides are in vitro transcribed RNA or synthetic RNA.

### Pharmaceutical Compositions

In another general aspect, the invention relates to a pharmaceutical composition comprising an isolated polynucleotide of the invention, an isolated polypeptide of the invention, a host cell of the invention, and/or an engineered immune cell of the invention and a pharmaceutically acceptable carrier. The term “pharmaceutical composition” as used herein means a product comprising an isolated polynucleotide of the invention, an isolated polypeptide of the invention, a host cell of the invention, and/or an engineered immune cell of the invention together with a pharmaceutically acceptable carrier. Polynucleotides, polypeptides, host cells, and/or engineered immune cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

### Methods of Use

In another general aspect, the invention relates to a method of treating a cancer in a subject in need thereof, comprising administering to the subject the CAR-T cells and/or CAR-NK cells of the invention. The cancer can, for example, be selected from but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of treating an inflammatory disease in a subject in need thereof, comprising administering to the subject the CAR-T cells and/or CAR-NK cells of the invention.

According to embodiments of the invention, the CAR-T cell or CAR-NK cell comprises a therapeutically effective amount of the expressed CARs of the invention. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to CARs, a therapeutically effective amount means an amount of the CAR molecule expressed in the transduced T cell or NK cell that modulates an immune response in a subject in need thereof. Also, as used herein with reference to CARs, a therapeutically effective amount means an amount of the CAR molecule expressed in the transduced T cell or NK cell that results in treatment of a disease, disorder, or condition; prevents or slows the progression of the disease, disorder, or condition; or reduces or completely alleviates symptoms associated with the disease, disorder, or condition.

As used herein with reference to CAR-T cell or CAR-NK cell, a therapeutically effective amount means an amount of the CAR-T cells or CAR-NK cells that modulates an immune response in a subject in need thereof. Also, as used herein with reference to CAR-T cell or CAR-NK cell, a therapeutically effective amount means an amount of the CAR-T cells or CAR-NK cells that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. According to other particular embodiments, the disease, disorder or condition to be treated is an inflammatory disease.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermally, intratumorally, intranodally, intramedullary, intramuscularly, intrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8 ×10⁷, 1×10⁵ to 7×10⁷, 1×10⁵ to ×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer and/or an inflammatory disease, disorder or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer and/or an inflammatory disease, disorder or condition. For cancer therapy, the provided compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti- FOLR1 mAb, an anti-CTLA-4 mAb, an anti-PD-L1 mAb, an anti-PD-1 mAb, other immuno- oncology drugs, an antiangiogenic agent, a radiation therapy, an antibody-drug conjugate (ADC), a targeted therapy, or other anticancer drugs.

According to particular embodiments, the methods of treating cancer and/or inflammatory disease in a subject in need thereof comprise administering to the subject the CAR-T cells and/or CAR-NK cells of the invention in combination with an agent that increases the efficacy of a cell expressing a CAR molecule. Such agents include, but are not limited to, an antibody fragment that binds to CD73, CD39, PD1, PD-L1, PD-L2, CTLA4, TIM3 or LAG3, or an adenosine A2a receptor antagonist.

According to particular embodiments, the methods of treating cancer and/or inflammatory disease in a subject in need thereof comprise administering to the subject the CAR-T cells and/or CAR-NK cells of the invention in combination with an agent that ameliorates one or more side effects associated with administration of a cell expressing a CAR molecule. Such agents include, but are not limited to, a steroid, an inhibitor of TNFα, or an inhibitor of IL-6.

According to particular embodiments, the methods of treating cancer and/or inflammatory disease in a subject in need thereof comprise administering to the subject the CAR-T cells and/or CAR-NK cells of the invention in combination with an agent that treats the disease associated with Claudin 18.2. Such agents include, but are not limited to, an anti-Claudin 18.2 monoclonal antibody or bispecific antibody.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### EMBODIMENTS

The invention provides also the following non-limiting embodiments.

Embodiment 1 is an isolated polynucleotide comprising a nucleic acid sequence encoding a chimeric antigen receptor (CAR), wherein the CAR comprises: (a) an extracellular domain comprising at least one antigen binding domain that specifically binds claudin 18.2 (CLDN18.2); (b) a hinge region; (c) a transmembrane region; and (d) an intracellular signaling domain.

Embodiment 2 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 21, 22, 23, 51, 52 and 53, respectively;
(2) SEQ ID NOs: 24, 25, 26, 54, 55 and 56, respectively;
(3) SEQ ID NOs: 27, 28, 29, 57, 58 and 59, respectively;
(4) SEQ ID NOs: 30, 31, 32, 60, 61 and 62, respectively;
(5) SEQ ID NOs: 33, 34, 35, 63, 64 and 65, respectively;
(6) SEQ ID NOs: 36, 37, 38, 66, 67 and 68, respectively;
(7) SEQ ID NOs: 39, 40, 41, 69, 70 and 71, respectively;
(8) SEQ ID NOs: 42, 43, 44, 72, 73 and 74, respectively;
(9) SEQ ID NOs: 45, 46, 47, 75, 76 and 77, respectively; or
(10) SEQ ID NOs: 48, 49, 50, 78, 79 and 80, respectively;
   wherein the antigen binding domain thereof specifically binds CLDN18.2, preferably human CLDN18.2.

Embodiment 3 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 81, 82, 83, 111, 112 and 113, respectively;
(2) SEQ ID NOs: 84, 85, 86, 114, 115 and 116, respectively;
(3) SEQ ID NOs: 87, 88, 89, 117, 118 and 119, respectively;
(4) SEQ ID NOs: 90, 91, 92, 120, 121 and 122, respectively;
(5) SEQ ID NOs: 93, 94, 95, 123, 124 and 125, respectively;
(6) SEQ ID NOs: 96, 97, 98, 126, 127 and 128, respectively;
(7) SEQ ID NOs: 99, 100, 101, 129, 130 and 131, respectively;
(8) SEQ ID NOs: 102, 103, 104, 132, 133 and 134, respectively;
(9) SEQ ID NOs: 105, 106, 107, 135, 136 and 137, respectively; or
(10) SEQ ID NOs: 108, 109, 110, 138, 139 and 140, respectively;
   wherein the antigen binding domain thereof specifically binds CLDN18.2, preferably human CLDN18.2.

Embodiment 4 is the isolated polynucleotide of any one of embodiments 1-3, wherein the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, 15, 17, or 19, or a light chain variable region having a polypeptide sequence at least 95%, at least 96%, at least 97%, at least 98%, or at least 99% identical to SEQ ID NO: 2, 4, 6, 8, 10, 12, 14, 16, 18, or 20.

Embodiment 5 is the isolated polynucleotide of any one of embodiments 1-4, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18; or
(10) a heavy chain variable region having the polypeptide sequence of SEQ ID NO:19, and a light chain variable region having the polypeptide sequence of SEQ ID NO:20.

Embodiment 6 is the isolated polynucleotide of any one of embodiments 1-3, wherein the antigen binding domain is humanized and comprises a heavy chain variable region having a polypeptide sequence at least 95%, at least 96%, at least 97%, at least 98%, or at least 99% identical to SEQ ID NO: 142, 143, 146, 147, 151, 152, 154, 155, 156, 159, 160, 161, 162, 166, 167, 170, 171, 172, 175, 176, 177, 178, 179, 180, 186, 187, 191, 192, or 193, or a light chain variable region having a polypeptide sequence at least 95%, at least 96%, at least 97%, at least 98%, or at least 99% identical to SEQ ID NO: 144, 145, 148, 149, 150, 153, 157, 158, 163, 164, 165, 168, 169, 173, 174, 181, 182, 183, 184, 185, 188, 189, 190, 194, 195, 196, or 197.

Embodiment 7 is the isolated polynucleotide of embodiment 6, wherein the antigen binding domain is humanized and comprises:
(1) a heavy chain variable region having the polypeptide sequence of SEQ ID NO:142, and a light chain variable region having the polypeptide sequence of SEQ ID NO:144;
(2) a heavy chain variable region having the polypeptide sequence of SEQ ID NO:142, and a light chain variable region having the polypeptide sequence of SEQ ID NO:145;
(3) a heavy chain variable region having the polypeptide sequence of SEQ ID NO:143, and a light chain variable region having the polypeptide sequence of SEQ ID NO:144;
(4) a heavy chain variable region having the polypeptide sequence of SEQ ID NO:143, and a light chain variable region having the polypeptide sequence of SEQ ID NO:145;
(5) a heavy chain variable region having the polypeptide sequence of SEQ ID NO:146, and a light chain variable region having the polypeptide sequence of SEQ ID NO:148;
(6) a heavy chain variable region having the polypeptide sequence of SEQ ID NO:146, and a light chain variable region having the polypeptide sequence of SEQ ID NO:149;
(7) a heavy chain variable region having the polypeptide sequence of SEQ ID NO:146, and a light chain variable region having the polypeptide sequence of SEQ ID NO:150;
(8) a heavy chain variable region having the polypeptide sequence of SEQ ID NO:147, and a light chain variable region having the polypeptide sequence of SEQ ID NO:148;
(9) a heavy chain variable region having the polypeptide sequence of SEQ ID NO:147, and a light chain variable region having the polypeptide sequence of SEQ ID NO:149;
(10) a heavy chain variable region having the polypeptide sequence of SEQ ID NO:147, and a light chain variable region having the polypeptide sequence of SEQ ID NO:150;
(11) a heavy chain variable region having the polypeptide sequence of SEQ ID NO:151, and a light chain variable region having the polypeptide sequence of SEQ ID NO:153;
(12) a heavy chain variable region having the polypeptide sequence of SEQ ID NO:152, and a light chain variable region having the polypeptide sequence of SEQ ID NO:153;
(13) a heavy chain variable region having the polypeptide sequence of SEQ ID NO:154, and a light chain variable region having the polypeptide sequence of SEQ ID NO:157;
(14) a heavy chain variable region having the polypeptide sequence of SEQ ID NO:155, and a light chain variable region having the polypeptide sequence of SEQ ID NO:157;
(15) a heavy chain variable region having the polypeptide sequence of SEQ ID NO:156, and a light chain variable region having the polypeptide sequence of SEQ ID NO:158;
(16) a heavy chain variable region having the polypeptide sequence of SEQ ID NO:159, and a light chain variable region having the polypeptide sequence of SEQ ID NO:163;
(17) a heavy chain variable region having the polypeptide sequence of SEQ ID NO:159, and a light chain variable region having the polypeptide sequence of SEQ ID NO:164;
(18) a heavy chain variable region having the polypeptide sequence of SEQ ID NO:160, and a light chain variable region having the polypeptide sequence of SEQ ID NO:163;
(19) a heavy chain variable region having the polypeptide sequence of SEQ ID NO:160, and a light chain variable region having the polypeptide sequence of SEQ ID NO:164;
(20) a heavy chain variable region having the polypeptide sequence of SEQ ID NO:161, and a light chain variable region having the polypeptide sequence of SEQ ID NO:165; or
(21) a heavy chain variable region having the polypeptide sequence of SEQ ID NO:162, and a light chain variable region having the polypeptide sequence of SEQ ID NO:165.

Embodiment 8 is the isolated polynucleotide of any one of embodiments 1-7, wherein the antigen binding domain is a single chain variable fragment (scFv) that specifically binds human CLDN18.2, preferably human CLDN18.2.

Embodiment 9 is the isolated polynucleotide of embodiment 8, wherein the single chain variable fragment (scFv) is humanized and comprises a polypeptide sequence at least 95% identical to any one of SEQ ID NOs: 198-215.

Embodiment 10 is the isolated polynucleotide of any one of embodiments 1-9, wherein the chimeric antigen receptor (CAR) comprise one or more antigen binding domains.

Embodiment 11 is the isolated polynucleotide of any one of embodiments 1-10, wherein the intracellular signaling domain of the CAR comprises one or more costimulatory domains and one or more activating domains.

Embodiment 12 is a chimeric antigen receptor (CAR) encoded by the isolated polynucleotide of any one of embodiments 1-11.

Embodiment 13 is a vector comprising the isolated polynucleotide of any one of embodiments 1-11.

Embodiment 14 is a host cell comprising the vector of embodiment 13.

Embodiment 15 is the host cell of embodiment 14, wherein the cell is a CAR-T cell, preferably a human CAR-T cell.

Embodiment 16 is the host cell of embodiment 14, wherein the cell is a CAR-NK cell, preferably a human CAR-NK cell.

Embodiment 17 is a method of making a host cell expressing a chimeric antigen receptor (CAR), the method comprising transducing a T cell with the vector of embodiment 13.

Embodiment 18 is a method of producing a chimeric antigen receptor (CAR)-T cell, the method comprising culturing T cells comprising the isolated polynucleotide comprising a nucleic acid encoding a chimeric antigen receptor (CAR) of any one of embodiments 1-11 under conditions to produce the CAR-T cell and recovering the CAR-T cell.

Embodiment 19 is a method of making a host cell expressing a chimeric antigen receptor (CAR), the method comprising transducing a NK cell with the vector of embodiment 13.

Embodiment 20 is a method of producing a chimeric antigen receptor (CAR)-NK cell, the method comprising culturing NK cells comprising the isolated polynucleotide comprising a nucleic acid encoding a chimeric antigen receptor (CAR) of any one of embodiments 1-11 under conditions to produce the CAR-NK cell, and recovering the CAR-NK cell.

Embodiment 21 is a method of generating a cell comprising a chimeric antigen receptor (CAR), the method comprising contacting a cell with the isolated polynucleotide comprising a nucleic acid encoding a chimeric antigen receptor (CAR) of any one of embodiments 1-11, wherein the isolated polynucleotide is an in vitro transcribed RNA or synthetic RNA.

Embodiment 22 is a method of treating cancer in a subject in need thereof, the method comprising administering to the subject the host cell of any one of embodiments 14-16.

Embodiment 23 is the method of embodiment 22, wherein the cancer is selected from a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 24 is a method of treating an inflammatory disease in a subject in need thereof, the method comprising administering to the subject the host cell of any one of embodiments 14-16.

Embodiment 25 is the method of any one of embodiments 22-24, further comprising administering to the subject in need thereof an agent that increases the efficacy of a cell expressing a CAR molecule.

Embodiment 26 is the method of any one of embodiments 22-24, further comprising administering to the subject in need thereof an agent that ameliorates one or more side effects associated with administration of a cell expressing a CAR molecule.

Embodiment 27 is the method of any one of embodiments 22-24, further comprising administering to the subject in need thereof an agent that treats the disease associated with Claudin 18.2.

### EXAMPLES

### Example 1: Identification of Antigen Binding Domains that Specifically Bind CLDN18.2

The antigen binding domains that specifically bind CLDN18.2 are anti-CLDN18.2 mAbs isolated and sequenced as described in PCT/US19/020872, filed on Mar. 6, 2019, which is incorporated herein by reference in its entirety.

Sequences of heavy and light chain variable regions for the antigen binding domains that specifically bind CLDN18.2 are provided in Tables 1 and 2, and the CDR regions for the antigen binding domains that specifically bind CLDN18.2 are provided in Tables 3-6.

TABLE 1
Sequences of heavy chain variable regions for the antigen
binding domains that specifically bind CLDN18.2

| | | SEQ ID |
|---|---|---|
| Name | VH | NO: |
| 2-C3 | EVQLVESGGDLVKPGGSLKLSCAASGFTFSSYGMSWVRQTPDKRLEWVA | 1 |
| | TISGGGSYTYYLDSVKGRFTISRDIAKNTLYLQMSSLKSEDTAMYFCARQS | |
| | RGNAMDYWGQGTSVTVSS | |
| 2-P8 | EVQLQQSGPELVKPGASVKMSCKASGYSFTGYNMHWVKQSHGKSLEWI | 3 |
| | GYIDPYNGVTNYNQKFKGKATLTVDKSSSTAYVQLNSLTSEDSAVYYCA | |
| | RWGGNYVDYWGQGTTLKVSS | |
| 3-E21 | EVQLVESGGALVKPGGSLKLSCAASGFTFSKYAMSWVRQTPEKRLEWVA | 5 |
| | FISNGGSYTYCLDSVKGRFTISRDNAKNTLYLQMSSLRSEDTALYYCARH | |
| | DKGNALDYWGQGNSVTVSS | |
| 3-P21 | EIQLQQSGAELVKPGASVKISCKASGYSFTGYNMKWVKQSHGKSLEWIG | 7 |
| | NINPYFGSTNYNQKFKGKATLTVDKSSSTAYMQLNSLTSEDSAVYYCARG | |
| | AYYGNAMDYWGQGTSVTVSS | |
| 5-E22 | KVQLQQSGPDLVEPGASVKISCKASGYTITDNYMHWVKQKPGQGLEWIG | 9 |
| | EIYPGSGNTYYNERFKGKATLTADKSSSTAYMQLSSLTSEDSAVYFCARG | |
| | FPYYAMDYWGPGTSVTVSS | |
| 6-J11 | DVQLVESGGGLVQPGGSRKLSCAASGFIFSSFGMHWVRQAPEKGLEWVA | 11 |
| | YISSGRSTMYYADTVKGRFTISRDNPKNTLFLQMTSLRSEDTAMYYCARG | |
| | GFYGNSLDYWGQGTSVTVSS | |
| 8-G12 | QVQLQQSGPELVKPGASVKISCKASGYAFSDYWMNWVKQRPGKGLEWI | 13 |
| | GQIYPGYGDTKYNENFKGTATLTADKSSSTAYMQLSSLTSEDSAVYFCAR | |
| | WGYYGNAMDYWGQGTSVTVSS | |
| 10-J10 | QVQLQQPGAELVKPGASVKLSCKASGYTFTRYRMNWVKQRPGQGLEWI | 15 |
| | GNIDPSDSETHYNQKFKDKATLTVDKSSSTAYMQLSSLTSEDSAVFYCAR | |
| | LNYGNCFDYWGQGTTLTVSS | |
| 10-K2 | EVQLQQSGPELVKPGASVKMSCKASGYAFTSYVMHWVKQKPGQGLEWI | 17 |
| | GYINPYSDGTRYNEKFKGKATLTSDKSSSTAYMELSSLTSEDSAVYYCTRI | |
| | YYGNAMDYWGQGTSVTVSS | |
| 15-D6 | QVQLQQPGADLVKPGASVKLSCKASGYTFTSYWINWVKQRPGQGLEWIG | 19 |
| | NIYPGRSSTNYNEKFKSKATLTVDTSSSTAYMQLSSLASDDSAVYYCSRLS | |
| | RGNAMDYWGQGTSVTVSS | |

VH: heavy chain variable region

TABLE 2
Sequences of light chain variable regions for the antigen binding
domains that specifically bind CLDN18.2

| | | SEQ |
|---|---|---|
| Name | VL | ID NO: |
| 2-C3 | DIVMTQSPSSLTVTAGEKVTMSCKSSQSLLNSGNQKNYLTWYQQKPGQPPKLLIY | 2 |
| | WASTRESGVPDRFTGSGSGTDFTLTISSVQAEDLAVYYCQNDYSYPLTFGAGTKLE | |
| | LK | |
| 2-P8 | DIKMTQSPSSMYASLGERVTITCKASQDINRYLSWFQQKPGKSPKTLIYRANRLVD | 4 |
| | GVPSRFSGSFSGQDYSLTISSLEYEDMGIYYCLQYDEFPLTFGAGTKLELK | |
| 3-E21 | DIVMTQSPSSLTVTAGEKVTMSCKSSQSLLNSGNQKNYLTWYQQKPGQPPKLLIY | 6 |
| | WASTRESGVPDRFTGSGSGTDFTLTISSVQAEDLSVYYCQNDYFYPLTFGAGTKLE | |
| | LK | |
| 3-P21 | DIVMTQSPSSLTVTAGGKVTMSCKSSQSLLNSGNQKNYLTWYQQKPGQPPKLLIY | 8 |
| | WASTRESGVPDRFSGSGSGTDFTLTISSVQAEDLAVYYCQNDYFYPLTFGAGTKLE | |
| | LK | |
| 5-E22 | DIQMNQSPSSLSASLGDTITITCHARQNINVWLSWYQQKSGNIPKLLIYKASNLHTG | 10 |
| | VPSRFSGSGSGTRFTLTISSLQPEDMATYYCQQGQNYPLTFGGGTKLEIK | |
| 6-J11 | DIVMTQSPSSLTVTAGEKVTMSCKSSLSLLNSGNQKNYLTWYQQKPGQPPKLLIY | 12 |
| | WASTRESGVPDRFTGSGSGTDFTLTISSMQAEDLAVYSCQNAYSYPLTFGAGTKLE | |
| | LK | |
| 8-G12 | DIVMTQSPSSLTVTAGEKVTMSCKSSQSLLNSGNQKNYLTWYQQKPGQPPKLLIY | 14 |
| | WASTRESGVPDRFTGSGSGTDFTLTISSVQTEDLAIYYCQNAYIYPLTFGAGTKLEL | |
| | K | |
| 10-J10 | DIVMTQSPSSLTVTAGEKVTMSCKSSQTLLNSGNQKNYLTWYQQKPGQPPKLLIY | 16 |
| | WASTRESGVPDRFTGSGSGTDFTLTISSVQAEDLAVFYCQNDYFYPFTFGSGTKLEI | |
| | K | |
| 10-K2 | DIVMTQSPSSLTVTAGEKVTMSCKSSQSLLNSGNQKNYLTWYQQKPGQPPKLLIY | 18 |
| | WASTRESGVPDRFTGSGSGTDFTLTISSVQAEDLAVYYCQNDYSYPFTFGSGTKLEI | |
| | K | |
| 15-D6 | DIVMTQSPSSLTVTAGEKVTMSCRSSQSLLNSGNQKSYLTWYQQKPGQPPKLLIYW | 20 |
| | ASTRESGVPDRFTGSGSGTDFTLTISSVQAEDLAVYYCQNDYYYPFTFGSGTKLEIK | |

VL: light chain variable region

TABLE 3
CDR regions 1-3 of heavy chain for the
antigen binding domains that specifically
bind CLDN18.2

| Name | HC CDR1 | NO | HC CDR2 | NO | HC CDR3 | NO |
|---|---|---|---|---|---|---|
| 2-C3 | GFTFSSYG | 21 | ISGGGSYT | 22 | ARQSRGNAMDY | 23 |
| 2-P8 | GYSFTGYN | 24 | IDPYNGVT | 25 | ARWGGNYVDY | 26 |
| 3-E21 | GFTFSKYA | 27 | ISNGGSYT | 28 | ARHDKGNALDY | 29 |
| 3-P21 | GYSFTGYN | 30 | INPYFGST | 31 | ARGAYYGNAMDY | 32 |
| 5-E22 | GYTITDNY | 33 | IYPGSGNT | 34 | ARGFPYYAMDY | 35 |
| 6-J11 | GFIFSSFG | 36 | ISSGRSTM | 37 | ARGGFYGNSLDY | 38 |
| 8-G12 | GYAFSDYW | 39 | IYPGYGDT | 40 | ARWGYYGNAMDY | 41 |
| 10-J10 | GYTFTRYR | 42 | IDPSDSET | 43 | ARLNYGNCFDY | 44 |
| 10-K2 | GYAFTSYV | 45 | INPYSDGT | 46 | TRIYYGNAMDY | 47 |
| 15-D6 | GYTFTSYW | 48 | IYPGRSST | 49 | SRLSRGNAMDY | 50 |

HC: heavy chain;
CDR: complementarity determining region;
NO: SEQ ID NO

The HC CDRs for the antigen binding domains that specifically bind CLDN18.2 were determined utilizing the IMGT method (Lefranc. M.-P. et al. Nucleic Acids Res 1999; 27:209-212).

TABLE 4
CDR regions 1-3 of light chain for the
antigen binding domains that specifically
bind CLDN18.2

| Name | LC CDR1 | NO | LC CDR2 | NO | LC CDR3 | NO |
|---|---|---|---|---|---|---|
| 2-C3 | QSLLNSGNQKNY | 51 | WAS | 52 | QNDYSYPLT | 53 |
| 2-P8 | QDINRY | 54 | RAN | 55 | LQYDEFPLT | 56 |
| 3-E21 | QSLLNSGNQKNY | 57 | WAS | 58 | QNDYFYPLT | 59 |
| 3-P21 | QSLLNSGNQKNY | 60 | WAS | 61 | QNDYFYPLT | 62 |
| 5-E22 | QNINVW | 63 | KAS | 64 | QQGQNYPLT | 65 |
| 6-J11 | LSLLNSGNQKNY | 66 | WAS | 67 | QNAYSYPLT | 68 |
| 8-G12 | QSLLNSGNQKNY | 69 | WAS | 70 | QNAYIYPLT | 71 |
| 10-J10 | QTLLNSGNQKNY | 72 | WAS | 73 | QNDYFYPFT | 74 |
| 10-K2 | QSLLNSGNQKNY | 75 | WAS | 76 | QNDYSYPFT | 77 |
| 15-D6 | QSLLNSGNQKSY | 78 | WAS | 79 | QNDYYYPFT | 80 |

LC: light chain;
CDR: complementarity determining region;
NO: SEQ ID NO

The LC CDRs for the antigen binding domains that specifically bind CLDN18.2 were determined utilizing the IMGT method (Lefranc. M.-P. et al. Nucleic Acids Res. 1999; 27:209-212).

TABLE 5
CDR regions 1-3 of heavy chain for the
antigen binding domains that specifically
bind CLDN18.2

| Name | HC CDR1 | NO | HC CDR2 | NO | HC CDR3 | NO |
|---|---|---|---|---|---|---|
| 2-C3 | GFTFSSYGMS | 81 | TISGGGSYTYYLDSVKG | 82 | ARQSRGNAMDY | 83 |
| 2-P8 | GYSFTGYNMH | 84 | YIDPYNGVTNYNQKFKG | 85 | ARWGGNYVDY | 86 |
| 3-E21 | GFTFSKYAMS | 87 | FISNGGSYTYCLDSVKG | 88 | ARHDKGNALDY | 89 |
| 3-P21 | GYSFTGYNMK | 90 | NINPYFGSTNYNQKFKG | 91 | ARGAYYGNAMDY | 92 |
| 5-E22 | GYTITDNYMH | 93 | EIYPGSGNTYYNERFKG | 94 | ARGFPYYAMDY | 95 |
| 6-J11 | GFIFSSFGMH | 96 | YISSGRSTMYYADTVKG | 97 | ARGGFYGNSLDY | 98 |
| 8-G12 | GYAFSDYWMN | 99 | QIYPGYGDTKYNENFKG | 100 | ARWGYYGNAMDY | 101 |
| 10-J10 | GYTFTRYRMN | 102 | NIDPSDSETHYNQKFKD | 103 | ARLNYGNCFDY | 104 |
| 10-K2 | GYAFTSYVMH | 105 | YINPYSDGTRYNEKFKG | 106 | TRIYYGNAMDY | 107 |
| 15-D6 | GYTFTSYWIN | 108 | NIYPGRSSTNYNEKFKS | 109 | SRLSRGNAMDY | 110 |

HC: heavy chain;
CDR: complementarity determining region;
NO: SEQ ID NO

The HC CDRs for (he antigen binding domains that specifically bind CLDN18.2 were determined utilizing a combination of IMGT (Lefranc, M.-P et al.. Nucleic Acids Res. 1999; 27:209-212) and Kabat (Elvin A. Kabat et al. Sequences of Proteins of Immunological Interest 5th ed. (1991)) methods.

TABLE 6
CDR regions 1-3 of light chain for the antigen
binding domains that specifically bind CLDN18.2

| Name | LC CDR1 | NO | LC CDR2 | NO | LC CDR3 | NO |
|---|---|---|---|---|---|---|
| 2-C3 | KSSQSLLNSGNQKNYLT | 111 | WASTRES | 112 | QNDYSYPLT | 113 |
| 2-P8 | KASQDINRYLS | 114 | RANRLVD | 115 | LQYDEFPLT | 116 |
| 3-E21 | KSSQSLLNSGNQKNYLT | 117 | WASTRES | 118 | QNDYFYPLT | 119 |
| 3-P21 | KSSQSLLNSGNQKNYLT | 120 | WASTRES | 121 | QNDYFYPLT | 122 |
| 5-E22 | HARQNINVWLS | 123 | KASNLHT | 124 | QQGQNYPLT | 125 |
| 6-J11 | KSSLSLLNSGNQKNYLT | 126 | WASTRES | 127 | QNAYSYPLT | 128 |
| 8-G12 | KSSQSLLNSGNQKNYLT | 129 | WASTRES | 130 | QNAYIYPLT | 131 |
| 10-J10 | KSSQTLLNSGNQKNYLT | 132 | WASTRES | 133 | QNDYFYPFT | 134 |
| 10-K2 | KSSQSLLNSGNQKNYLT | 135 | WASTRES | 136 | QNDYSYPFT | 137 |
| 15-D6 | RSSQSLLNSGNQKSYLT | 138 | WASTRES | 139 | QNDYYYPFT | 140 |

LC: light chain;
CDR: complementarity determining region;
NO: SEQ ID NO

The LC CDRs for the antigen binding domains that specifically bind CLDN18.2 were detemiined utilizing a combination of IMGT (Lefranc. M.-P et al.. Nucleic Acids Res. 1999; 27:209-212) and Kabat (Elvin A. Kabat et al. Sequences of Proteins of Immunological Interest 5th ed. (1991)) methods.

### Example 2: Humanization of Mouse Anti-CLDN18.2 mAbs

The mouse anti-CLDN18.2 mAbs were humanized to reduce the potential of immunogenicity when used in human patients as described in PCT/US19/020872, filed on Mar. 6, 2019, which is incorporated herein by reference in its entirety. The sequences of the humanized VH and VL regions are shown in Table 7.

TABLE 7
Sequences of heavy chain and light chain variable regions of
humanized antigen binding domains that specifically bind CLDN18.2

| | | SEQ |
|---|---|---|
| | | ID |
| VH/VL | SEQUENCE | NO: |
| 2-C3-H1 | QVTLRESGPALVKPTQTLTLTCTASGFTFSSYGMSWVRQPPGKALEWVATISGGGS | 142 |
| | YTYYNPSLKDRFTISRDISANQLVLKVTNMDPADTATYFCARQSRGNAMDYWGQG | |
| | TTVTVSS | |
| 2-C3-H2 | QVTLRESGPALVKPTQTLTLTCTFSGFTFSSYGMSWIRQPPGKALEWLATISGGGSY | 143 |
| | TYYLDSLKDRFTISRDISKNQVVLTVTNMDPADTATYFCARQSRGNAMDYWGQGT | |
| | TVTVSS | |
| 2-C3-L1 | DIQMTQSPSTLSASVGDRVTITCKSSQSLLNSGNQKNYLTWYQQKPGKAPKLLIYW | 144 |
| | ASTRESGVPSRFSGSGSGTAFTLTISSLQPDDFATYYCQNDYSYPLTFGGGTKVEIK | |
| 2-C3-L2 | DIQMTQSPSTLSASVGDRVTITCKSSQSLLNSGNQKNYLTWYQQKPGKAPKLLIYW | 145 |
| | ASTRESGVPSRFSGSGSGTEFTLTISSLQPDDFATYYCQNDYSYPLTFGGGTKVEIK | |
| 5-E22-H1 | QVQLVQSGVEVKKPGASVKVSCKASGYTITDNYMHWVRQAPGQGLEWIGEIYPGS | 146 |
| | GNTYFNEKFKNRATLTADKSTTTAYMELKSLQFDDTAVYFCARGFPYYAMDYWG | |
| | QGTTVTVSS | |
| 5-E22-H3 | QVQLVQSGAEVKKPGASVKVSCKASGYTITDNYMHWVRQAPGQGLEWIGEIYPGS | 147 |
| | GNTYYAEKFKNRATLTADKSISTAYMELSRLRSDDTAVYFCARGFPYYAMDYWGQ | |
| | GTLVTVSS | |
| 5-E22-L1 | EIVMTQSPATLSLSPGERATLSCHARQNINVWLSWYQQKPGQAPRLLIYKASNLHT | 148 |
| | GVPARFSGSGSGTDFTLTISSLEPEDFAVYYCQQGQNYPLTFGGGTKVEIK | |
| 5-E22-L2 | EIVLTQSPATLSLSPGERATLSCHARQNINVWLSWYQQKPGQAPRLLIYKASNLHTG | 149 |
| | IPARFSGSGSGTDFTLTISSLEPEDFAVYYCQQGQNYPLTFGGGTKVEIK | |
| 5-E22-L3 | DIVMTQSPLSLPVTPGEPASISCHARQNINVWLSWYLQKPGQSPQLLIYKASNLHTG | 150 |
| | VPDRFSGSGSGTDFTLKISRVEAEDVGVYYCQQGQNYPLTFGQGTKVEIK | |
| 6-J11-H1 | EVQLVESGGGLVQPGGSLRLSCAASGFIFSSFGMHWVRQAPGKGLEWVAYISSGRS | 151 |
| | TMYYADSVKGRFTISRDNSKNTLYLQMNSLTAEDTAVYYCARGGFYGNSLDYWG | |
| | QGTLVTVSS | |
| 6-J11-H2 | EVQLVESGGGLVQPGGSLRLSCAASGFIFSSFGMHWVRQAPGKGLEWVAYISSGRS | 152 |
| | TMYYADSVKGRFTISRDNSKNTLYLQMNSLRSEDTAVYYCARGGFYGNSLDYWG | |
| | QGTLVTVSS | |
| 6-J11-L1 | DIQMTQSPSSLSASVGDRVTITCKSSLSLLNSGNQKNYLTWYQQKPGKAPKLLIYW | 153 |
| | ASTRESGVPSRFSGSGSGTDFTLTISSLQPEDFATYSCQNAYSYPLTFGQGTKVEIK | |
| 3-E21-H1 | QVQLQESGPGLVRPSQTLSLTCTASGFTFSKYAMNWVRQPPGRGLEWVAFISNGGS | 154 |
| | YTEYNPSVKGRFTILRDNSKNQLSLRLSSVTAADTAVYYCARHDKGNALDYWGQG | |
| | SLVTVSS | |
| 3-E21-H2 | QVQLQESGPGLVRPSQTLSLTCTASGFTFSKYAMSWVRQPPGRGLEWVAFISNGGS | 155 |
| | YTEYNPSVKGRFTILRDNSKNQLSLKLSSVTAADTAVYYCARHDKGNALDYWGQG | |
| | SLVTVSS | |
| 3-E21-H3 | EVQLLESGGGLVQPGGSLRLSCAASGFTFSKYAMSWVRQAPGKGLEWVAAISNGG | 156 |
| | SYTYYADSVKGRFTISRDNSKNTLYLQMNSLRAEDTAVYYCARHDKGNALDYWG | |
| | QGTLVTVSS | |
| 3-E21-L1 | DIQMTQSPSSLSASVGDRVTITCKSSQSLLNSGNQKNYLTWYQQKPGKAPKLLIYW | 157 |
| | ASNLQTGVPSRFSGSGSGTDFTFTISSLQPEDIATYYCQNDYFYPLTFGQGTKVEIK | |
| 3-E21-L2 | DIVMTQSPDSLAVSLGERATINCKSSQSLLNSGNQKNYLTWYQQKPGQPPKLLIYW | 158 |
| | ASTRESGVPDRFSGSGSGTDFTLTISSLQAEDVAVYYCQNDYFYPLTFGQGTRLEIK | |
| 3-P21-H1 | EIQLVESGGGLVQPGGSLRLSCAASGYSFTGYNIHWVRQAPGKGLEWIGYINPYFGS | 159 |
| | TDYADSVKGRATLSVDKSKNTAYLQMNSLRAEDTAVYYCARGAYYGNAMDYWG | |
| | QGTLVTVSS | |
| 3-P21-H2 | EIQLVESGGGLVQPGGSLRLSCAASGYSFTGYNMKWVRQAPGKGLEWIGNINPYFG | 160 |
| | STNYADSVKGRATLSVDKSKNTAYLQMNSLRAEDTAVYYCARGAYYGNAMDYW | |
| | GQGTLVTVSS | |
| 3-P21-H3 | EIQLVQSGAEVKKPGESLKISCKASGYSFTGYNIGWVRQMPGKGLEWIGIINPYFGS | 161 |
| | TRYSPSFQGQATLSVDKSISTAYLQWSSLKASDTAMYYCARGAYYGNAMDYWGQ | |
| | GTLVTVSS | |
| 3-P21-H4 | EIQLVQSGAEVKKPGESLKISCKASGYSFTGYNMKWVRQMPGKGLEWIGIINPYFGS | 162 |
| | TNYSPSFQGQATLSVDKSISTAYLQWSSLKASDTAMYYCARGAYYGNAMDYWGQ | |
| | GTLVTVSS | |
| 3-P21-L1 | DIQMTQSPSSLSASVGDRVTITCRSSQSLLNSGNQKNYVTWYQQKPGKAPKLLIYW | 163 |
| | ASFLYSGVPSRFSGSGSGTDFTLTISSLQPEDFATYYCQNDYFYPLTFGQGTKVEIK | |
| 3-P21-L2 | DIQMTQSPSSLSASVGDRVTITCRSSQSLLNSGNQKNYVTWYQQKPGKAPKLLIYW | 164 |
| | ASTRESGVPSRFSGSGSGTDFTLTISSLQPEDFATYYCQNDYFYPLTFGQGTKVEIK | |
| 3-P21-L3 | DIVMTQSPDSLAVSLGERATINCKSSQSLLNSGNQKNYLTWYQQKPGQPPKLLIYW | 165 |
| | ASTRESGVPDRFSGSGSGTDFTLTISSLQAEDVAVYYCQNDYFYPLTFGQGTKVEIK | |
| 8-G12-H1 | EVQLVESGGGLVQPGGSLRLSCAASGYAFSDYWMNWVRQAPGKGLEWIGQIYPG | 166 |
| | YGDTKHNQRFMDRATLSADKSTSTAYMQMNSLRAEDTAVYFCARWGYYGNAMD | |
| | YWGQGTLVTVSS | |
| 8-G12-H2 | QVQLVQSGAEVKKPGASVKVSCKASGYAFSDYWMNWVRQAPGQGLEWIGQIYPG | 167 |
| | YGDTKYAQKFQGRATLTADKSISTAYMELSRLRSDDTAVYFCARWGYYGNAMDY | |
| | WGQGTLVTVSS | |
| 8-G12-L1 | DIQMTQSPSSLSASVGDRVTITCKSSQSLLNSGNQKNYLTWYQQKPGKAPKLLIYW | 168 |
| | ASTRESGVPSRFSGSGSGTDFTLTISSLQPEDFATYYCQNAYIYPLTFGQGTKVEIK | |
| 8-G12-L2 | DIVMTQSPDSLAVSLGERATINCKSSQSLLNSGNQKNYLTWYQQKPGQPPKLLIYW | 169 |
| | ASTRESGVPDRFSGSGSGTDFTLTISSLQAEDVAVYYCQNAYIYPLTFGGGTKVEIK | |
| 10-K2-H1 | EVQLVESGGGLVQPGGSLRLSCAASGYAFTSYVMHWVRQAPGKGLEWIGYINPYS | 170 |
| | DGTRHNQRFMDRATLSSDKSTSTAYMQMNSLRAEDTAVYYCTRIYYGNAMDYWG | |
| | QGTLVTVSS | |
| 10-K2-H2 | QVQLVQSGAEVRKPGASVTVSCKASGYAFTSYVMHWVRQAPGQGLEWIGYINPYS | 171 |
| | DGTRFAQKFKGRATLTSDKSTSTAFMELSSLRSDDTAIYYCTRIYYGNAMDYWGQ | |
| | GTLVTVSS | |
| 10-K2-H3 | QVQLVQSGAEVKKPGASVKVSCKASGYAFTSYVMHWVRQAPGQGLEWIGYINPY | 172 |
| | SDGTRFAQKFKGRVTLTSDKSTSTAYMELSSLRSDDTAVYYCTRIYYGNAMDYWG | |
| | QGTLVTVSS | |
| 10-K2-L1 | DIQMTQSPSSLSASVGDRVTITCKSSQSLLNSGNQKNYLTWYQQKPGKAPKLLIYW | 173 |
| | ASTRESGVPSRFSGSGSGTDFTLTISSLQPEDFATYYCQNDYSYPFTFGQGTKVEIK | |
| 10-K2-L2 | DIVMTQSPLSLPVTPGEAASISCKSSQSLLNSGNQKNYLTWYLQKPGQSPQLLIYWA | 174 |
| | STRESGVPHRFSGSGSGTEFTLKISRVEAEDVGVYYCQNDYSYPFTFGQGTKVEIK | |
| 15-D6-H1 | EVQLVESGGGLVQPGGSLRLSCAASGYTFTSYWINWVRQAPGKGLEWIGDIYPGRS | 175 |
| | STNYNQNFKDRATLSVDTSKNTAYLQMNSLRAEDTAVYYCSRLSRGNAMDYWGQ | |
| | GTLVTVSS | |
| 15-D6-H2 | EVQLVESGGGLVQPGGSLRLSCAASGYTFTSYWINWVRQAPGKGLEWIGDIYPGRS | 176 |
| | STNYNQNFKGRATLSVDTSKNTAYLQMNSLRAEDTAVYYCSRLSRGNAMDYWGQ | |
| | GTLVTVSS | |
| 15-D6-H3 | EVQLVESGGGLVQPGGSLRLSCAASGYTFTSYWINWVRQAPGKGLEWIGNIYPGRS | 177 |
| | STNYNQNFKGRATLSVDTSKNTAYLQMNSLRAEDTAVYYCSRLSRGNAMDYWGQ | |
| | GTLVTVSS | |
| 15-D6-H4 | EVQLVESGGGLVQPGGSLRLSCAASGYTFTSYWIHWVRQAPGKGLEWIGYIYPGRS | 178 |
| | STNYNEKFKGRATLSVDTSKNTAYLQMNSLRAEDTAVYYCSRLSRGNAMDYWGQ | |
| | GTLVTVSS | |
| 15-D6-H5 | EVQLVESGGGLVQPGGSLRLSCAASGYTFTSYWINWVRQAPGKGLEWIGYIYPGRS | 179 |
| | STNYNEKFKGRATLSVDTSKNTAYLQMNSLRAEDTAVYYCSRLSRGNAMDYWGQ | |
| | GTLVTVSS | |
| 15-D6-H6 | EVQLVESGGGLVQPGGSLRLSCAASGYTFTSYWINWVRQAPGKGLEWIGNIYPGRS | 180 |
| | STNYNEKFKGRATLSVDTSKNTAYLQMNSLRAEDTAVYYCSRLSRGNAMDYWGQ | |
| | GTLVTVSS | |
| 15-D6-L1 | DIQMTQSPSSLSASVGDRVTITCRSSQSLLNSGNQKSYMTWYQQKPGKAPKLLIYW | 181 |
| | ASNHASGVPSRFSGSGSGTDFTLTISSLQPEDFATYYCQNDYYYPFTFGQGTKVEIK | |
| 15-D6-L2 | DIQMTQSPSSLSASVGDRVTITCRSSQSLLNSGNQKSYMTWYQQKPGKAPKLLIYW | 182 |
| | ASTRESGVPSRFSGSGSGTDFTLTISSLQPEDFATYYCQNDYYYPFTFGQGTKVEIK | |
| 15-D6-L3 | DIQMTQSPSSLSASVGDRVTITCRSSQSLLNSGNQKSYLTWYQQKPGKAPKLLIYWA | 183 |
| | SNHASGVPSRFSGSGSGTDFTLTISSLQPEDFATYYCQNDYYYPFTFGQGTKVEIK | |
| 15-D6-L4 | DIQMTQSPSSLSASVGDRVTITCRSSQSLLNSGNQKSYVTWYQQKPGKAPKLLIYW | 184 |
| | ASHRYTGVPSRFSGSGSGTDFTLTISSLQPEDFATYYCQNDYYYPFTFGQGTKVEIK | |
| 15-D6-L5 | DIQMTQSPSSLSASVGDRVTITCRSSQSLLNSGNQKSYVTWYQQKPGKAPKLLIYW | 185 |
| | ASHRYTGVPSRFSGSGSGTEFTLTISSLQPEDFATYYCQNDYYYPFTFGQGTKVEIK | |
| 10-J10-H1 | QVQLVQSGAEVKKPGSSVKVSCKASGYTFTRYRISWVRQAPGQGLEWIGGIDPSDS | 186 |
| | ETNYAQKFQGRATLTVDKSTSTAYMELSSLRSEDTAVYYCARLNYGNCFDYWGQ | |
| | GTLVTVSS | |
| 10-J10-H2 | QVQLVQSGAEVKKPGSSVKVSCKASGYTFTRYRISWVRQAPGQGLEWIGGIDPSDS | 187 |
| | ETNYAQKFQGRATLTADKSTSTAYMELSSLRSEDTAVYYCARLNYGNCFDYWGQ | |
| | GTLVTVSS | |
| 10-J10-L1 | DIVMTQSPDSLAVSLGERATINCKSSQTLLNSGNQKNYLTWYQQKPGQPPKLLIYW | 188 |
| | ASTRESGVPDRFSGSGSGTDFTLTISSLQAEDVAVYYCQNDYFYPFTFGQGTRLEIK | |
| 10-J10-L2 | DIVMTQSPDSLAVSLGERATINCKSSQTLLNSGNQKNYLAWYQQKPGQPPKLLIYW | 189 |
| | ASTRESGVPDRFSGSGSGTDFTLTISSLQAEDVAVYYCQNDYFYPFTFGQGTKVEIK | |
| 10-J10-L3 | DIVMTQSPDSLAVSLGERATINCKSSQTLLNSGNQKNYLTWYQQKPGQPPKLLIYW | 190 |
| | ASTRESGVPDRFSGSGSGTDFTLTISSLQAEDVAVYYCQNDYFYPFTFGQGTKVEIK | |
| 2-P8-H1 | QVQLVQSGAEVKKPGSSVKVSCKASGYSFTGYNLHWVRQAPGQGLEWIGWIDPYN | 191 |
| | GVTQYNEKFKGRATLTVDKSTSTAYMELSSLRSEDTAVYYCARWGGNYVDYWGQ | |
| | GTTVTVSS | |
| 2-P8-H2 | QVQLVQSGAEVKKPGASVKVSCKASGYSFTGYNLHWVRQAPGQGLEWIGWIDPY | 192 |
| | NGVTQYNEKFKGRVTITVDKSTSTAYMELSSLRSEDTAVYYCARWGGNYVDYWG | |
| | QGTTVTVSS | |
| 2-P8-H3 | QVQLVQSGAEVKKPGSSVKVSCKASGYSFTGYNINWVRQAPGQGLEWIGWIDPYN | 193 |
| | GVTKYNEKFKGRATLTVDKSTNTAYMELSSLRSEDTAFYYCARWGGNYVDYWGQ | |
| | GTLVTVSS | |
| 2-P8-L1 | DIQMTQSPSSLSASVGDRVTITCRASQDINRYVSWFQQKPGKAPKTLIYRANYRYSG | 194 |
| | VPSRFSGSFSGQDYTLTISSLQPEDFATYYCLQYDEFPLTFGQGTKVEIK | |
| 2-P8-L2 | DIQMTQSPSSLSASVGDRVTITCKASQDINRYVSWFQQKPGKAPKTLIYRANYRYSG | 195 |
| | VPSRFSGSFSGQDYTLTISSLQPEDFATYYCLQYDEFPLTFGQGTKVEIK | |
| 2-P8-L3 | DIQMTQSPSSLSASVGDRVTITCKASQDINRYVSWFQQKPGKAPKSLIYRANYRYSG | 196 |
| | VPSRFSGSGSGQDYTLTISSLQPEDFATYYCLQYDEFPLTFGQGTKVEIK | |
| 2-P8-L4 | DIQMTQSPSTLSASVGDRVTITCRASQDINRYLSWFQQKPGKAPKTLIYRANNLASG | 197 |
| | VPSRFSGSFSGQEYTLTISSLQPDDFATYYCLQYDEFPLTFGQGTKVEIK | |

The humanized VH and VL regions were fused to the constant regions of human IgG1 heavy chain and kappa light chain, respectively. The humanized mAbs were named as follows: 2-C3-H1L1 refers to the mAb with the 2-C3-H1 heavy chain variable region and the 2-C3-L1 light chain variable region; all the other humanized mAbs adopt the same naming rule.

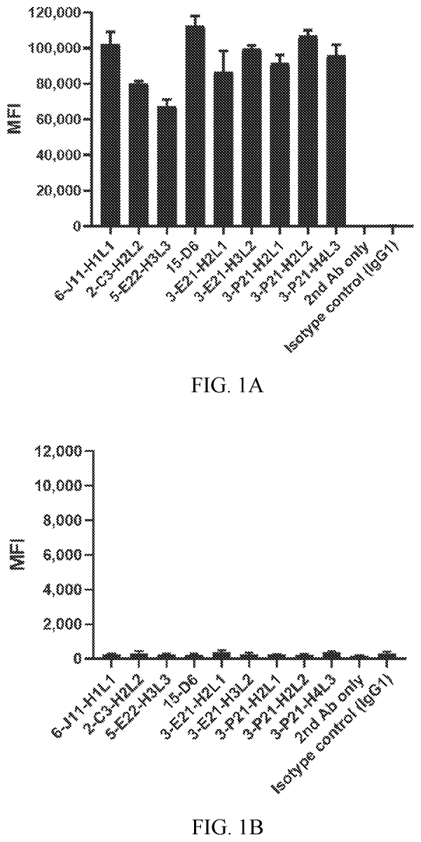

Several humanized mAbs were tested for their ability to bind CLDN18.2 and CLDN18.1. Chimeric mAb 15-D6 was also used in the assay. Stable cell lines (HEK293-CLDN18.2 and HEK293-CLDN18.1) expressing human CLDN18.2 and CLDN18.1, respectively, were used in FACS experiments with Alexa Fluor® 488-based detection as described in PCT/US19/020872. The mAbs were tested at 10 μg/mL. The results are shown in FIGS. 1A-1B. “MFI” is “Mean Fluorescence Intensity”.

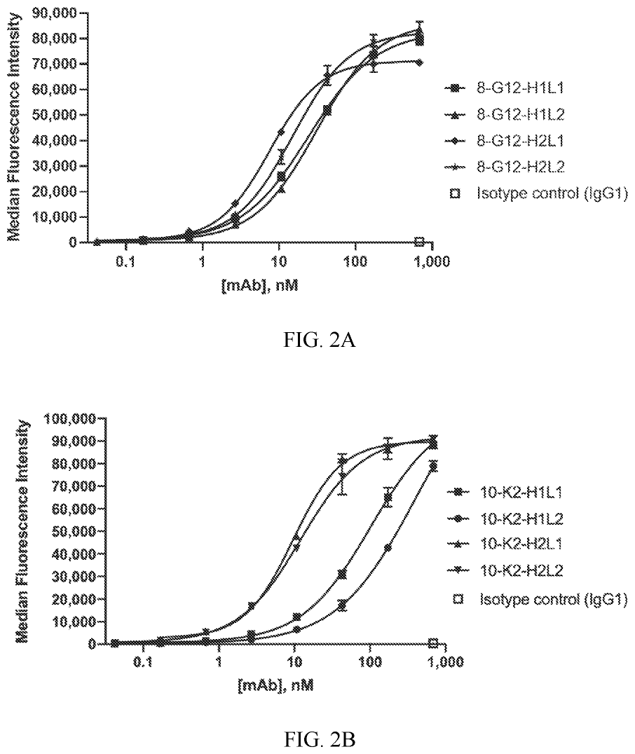

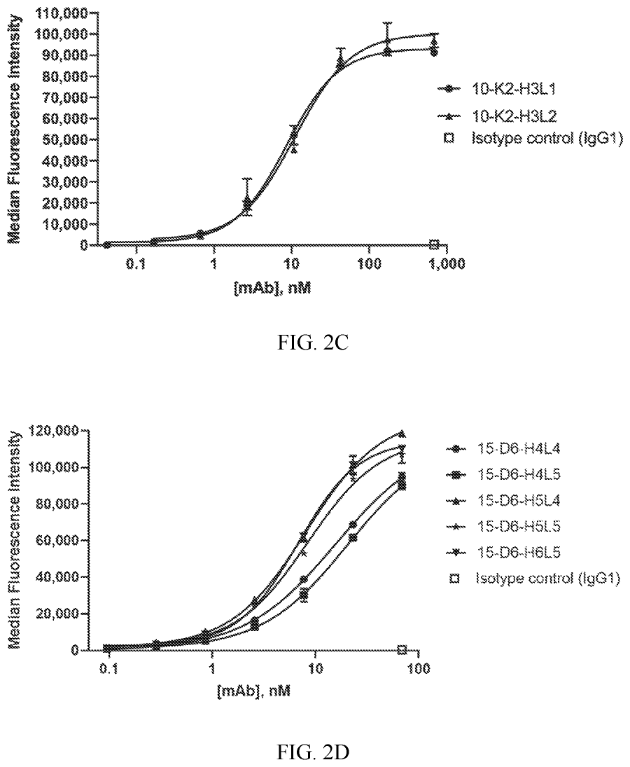

Additional humanized mAbs were tested for their ability to bind CLDN18.2 using the HEK293-CLDN18.2 stable cell line and the same FACS protocol, with the modification that propidium iodide (PI) was incubated together with the secondary antibody to label dead cells. The results are shown in FIGS. 2A-2D.

### Example 3: Conversion of Humanized mAbs to scFvs

The humanized mAbs were converted to scFvs, each of which consists of one VH and one VL with a (G₄S)ₙ linker in between (where “n” represents the number of the G₄S repeats). Either the VH or the VL region was placed at the N-terminus of the fusion protein to identify the most effective scFv designs. The sequences of the designed scFvs are shown in Table 8. The scFvs were named as following: 2-C3-H2(G₄S)₃L2 refers to the scFv with 2-C3-H2 heavy chain variable region, the (G₄S)₃ linker and 2-C3-L2 light chain variable region; all the other scFvs adopted the same naming rule.

TABLE 8
Sequences of humanized scFvs that specifically bind CLDN18.2

| | | SEQ |
|---|---|---|
| | | ID |
| Name | SEQUENCE | NO: |
| 2-C3- | QVTLRESGPALVKPTQTLTLTCTFSGFTFSSYGMSWIRQPPGKALEWLATISGGGS | 198 |
| H2(G₄S)₃L2 | YTYYLDSLKDRFTISRDISKNQVVLTVTNMDPADTATYFCARQSRGNAMDYWGQ | |
| | GTTVTVSSGGGGSGGGGSGGGGSDIQMTQSPSTLSASVGDRVTITCKSSQSLLNSG | |
| | NQKNYLTWYQQKPGKAPKLLIYWASTRESGVPSRFSGSGSGTEFTLTISSLQPDDF | |
| | ATYYCQNDYSYPLTFGGGTKVEIK | |
| 2-C3- | QVTLRESGPALVKPTQTLTLTCTFSGFTFSSYGMSWIRQPPGKALEWLATISGGGS | 199 |
| H2(G₄S)₄L2 | YTYYLDSLKDRFTISRDISKNQVVLTVTNMDPADTATYFCARQSRGNAMDYWGQ | |
| | GTTVTVSSGGGGSGGGGSGGGGSGGGGSDIQMTQSPSTLSASVGDRVTITCKSSQ | |
| | SLLNSGNQKNYLTWYQQKPGKAPKLLIYWASTRESGVPSRFSGSGSGTEFTLTISS | |
| | LQPDDFATYYCQNDYSYPLTFGGGTKVEIK | |
| 2-C3- | DIQMTQSPSTLSASVGDRVTITCKSSQSLLNSGNQKNYLTWYQQKPGKAPKLLIY | 200 |
| L2(G₄S)₃H2 | WASTRESGVPSRFSGSGSGTEFTLTISSLQPDDFATYYCQNDYSYPLTFGGGTKVEI | |
| | KGGGGSGGGGSGGGGSQVTLRESGPALVKPTQTLTLTCTFSGFTFSSYGMSWIRQ | |
| | PPGKALEWLATISGGGSYTYYLDSLKDRFTISRDISKNQVVLTVTNMDPADTATY | |
| | FCARQSRGNAMDYWGQGTTVTVSS | |
| 6-J11- | EVQLVESGGGLVQPGGSLRLSCAASGFIFSSFGMHWVRQAPGKGLEWVAYISSGR | 201 |
| H1(G₄S)₃L1 | STMYYADSVKGRFTISRDNSKNTLYLQMNSLTAEDTAVYYCARGGFYGNSLDY | |
| | WGQGTLVTVSSGGGGSGGGGSGGGGSDIQMTQSPSSLSASVGDRVTITCKSSLSL | |
| | LNSGNQKNYLTWYQQKPGKAPKLLIYWASTRESGVPSRFSGSGSGTDFTLTISSLQ | |
| | PEDFATYSCQNAYSYPLTFGQGTKVEIK | |
| 6-J11- | EVQLVESGGGLVQPGGSLRLSCAASGFIFSSFGMHWVRQAPGKGLEWVAYISSGR | 202 |
| H1(G₄S)₄L1 | STMYYADSVKGRFTISRDNSKNTLYLQMNSLTAEDTAVYYCARGGFYGNSLDY | |
| | WGQGTLVTVSSGGGGSGGGGSGGGGSGGGGSDIQMTQSPSSLSASVGDRVTITC | |
| | KSSLSLLNSGNQKNYLTWYQQKPGKAPKLLIYWASTRESGVPSRFSGSGSGTDFT | |
| | LTISSLQPEDFATYSCQNAYSYPLTFGQGTKVEIK | |
| 6-J11- | DIQMTQSPSSLSASVGDRVTITCKSSLSLLNSGNQKNYLTWYQQKPGKAPKLLIY | 203 |
| L1(G₄S)₃H1 | WASTRESGVPSRFSGSGSGTDFTLTISSLQPEDFATYSCQNAYSYPLTFGQGTKVEI | |
| | KGGGGSGGGGSGGGGSEVQLVESGGGLVQPGGSLRLSCAASGFIFSSFGMHWVR | |
| | QAPGKGLEWVAYISSGRSTMYYADSVKGRFTISRDNSKNTLYLQMNSLTAEDTA | |
| | VYYCARGGFYGNSLDYWGQGTLVTVSS | |
| 5-E22- | QVQLVQSGAEVKKPGASVKVSCKASGYTITDNYMHWVRQAPGQGLEWIGEIYP | 204 |
| H3(G₄S)₃L3 | GSGNTYYAEKFKNRATLTADKSISTAYMELSRLRSDDTAVYFCARGFPYYAMDY | |
| | WGQGTLVTVSSGGGGSGGGGSGGGGSDIVMTQSPLSLPVTPGEPASISCHARQNI | |
| | NVWLSWYLQKPGQSPQLLIYKASNLHTGVPDRFSGSGSGTDFTLKISRVEAEDVG | |
| | VYYCQQGQNYPLTFGQGTKVEIK | |
| 5-E22- | QVQLVQSGAEVKKPGASVKVSCKASGYTITDNYMHWVRQAPGQGLEWIGEIYP | 205 |
| H3(G₄S)₄L3 | GSGNTYYAEKFKNRATLTADKSISTAYMELSRLRSDDTAVYFCARGFPYYAMDY | |
| | WGQGTLVTVSSGGGGSGGGGSGGGGSGGGGSDIVMTQSPLSLPVTPGEPASISCH | |
| | ARQNINVWLSWYLQKPGQSPQLLIYKASNLHTGVPDRFSGSGSGTDFTLKISRVE | |
| | AEDVGVYYCQQGQNYPLTFGQGTKVEIK | |
| 5-E22- | DIVMTQSPLSLPVTPGEPASISCHARQNINVWLSWYLQKPGQSPQLLIYKASNLHT | 206 |
| L3(G₄S)₃H3 | GVPDRFSGSGSGTDFTLKISRVEAEDVGVYYCQQGQNYPLTFGQGTKVEIKGGGG | |
| | SGGGGSGGGGSQVQLVQSGAEVKKPGASVKVSCKASGYTITDNYMHWVRQAPG | |
| | QGLEWIGEIYPGSGNTYYAEKFKNRATLTADKSISTAYMELSRLRSDDTAVYFCA | |
| | RGFPYYAMDYWGQGTLVTVSS | |
| 3-E21- | EVQLLESGGGLVQPGGSLRLSCAASGFTFSKYAMSWVRQAPGKGLEWVAAISNG | 207 |
| H3(G₄S)₃L2 | GSYTYYADSVKGRFTISRDNSKNTLYLQMNSLRAEDTAVYYCARHDKGNALDY | |
| | WGQGTLVTVSSGGGGSGGGGSGGGGSDIVMTQSPDSLAVSLGERATINCKSSQSL | |
| | LNSGNQKNYLTWYQQKPGQPPKLLIYWASTRESGVPDRFSGSGSGTDFTLTISSLQ | |
| | AEDVAVYYCQNDYFYPLTFGQGTRLEIK | |
| 3-E21- | EVQLLESGGGLVQPGGSLRLSCAASGFTFSKYAMSWVRQAPGKGLEWVAAISNG | 208 |
| H3(G₄S)₄L2 | GSYTYYADSVKGRFTISRDNSKNTLYLQMNSLRAEDTAVYYCARHDKGNALDY | |
| | WGQGTLVTVSSGGGGSGGGGSGGGGSGGGGSDIVMTQSPDSLAVSLGERATINC | |
| | KSSQSLLNSGNQKNYLTWYQQKPGQPPKLLIYWASTRESGVPDRFSGSGSGTDFT | |
| | LTISSLQAEDVAVYYCQNDYFYPLTFGQGTRLEIK | |
| 3-E21- | DIVMTQSPDSLAVSLGERATINCKSSQSLLNSGNQKNYLTWYQQKPGQPPKLLIY | 209 |
| L2(G₄S)₃H3 | WASTRESGVPDRFSGSGSGTDFTLTISSLQAEDVAVYYCQNDYFYPLTFGQGTRL | |
| | EIKGGGGSGGGGSGGGGSEVQLLESGGGLVQPGGSLRLSCAASGFTFSKYAMSW | |
| | VRQAPGKGLEWVAAISNGGSYTYYADSVKGRFTISRDNSKNTLYLQMNSLRAED | |
| | TAVYYCARHDKGNALDYWGQGTLVTVSS | |
| 3-P21- | EIQLVESGGGLVQPGGSLRLSCAASGYSFTGYNMKWVRQAPGKGLEWIGNINPYF | 210 |
| H2(G₄S)₃L1 | GSTNYADSVKGRATLSVDKSKNTAYLQMNSLRAEDTAVYYCARGAYYGNAMD | |
| | YWGQGTLVTVSSGGGGSGGGGSGGGGSDIQMTQSPSSLSASVGDRVTITCRSSQS | |
| | LLNSGNQKNYVTWYQQKPGKAPKLLIYWASFLYSGVPSRFSGSGSGTDFTLTISSL | |
| | QPEDFATYYCQNDYFYPLTFGQGTKVEIK | |
| 3-P21- | EIQLVESGGGLVQPGGSLRLSCAASGYSFTGYNMKWVRQAPGKGLEWIGNINPYF | 211 |
| H2(G₄S)₄L1 | GSTNYADSVKGRATLSVDKSKNTAYLQMNSLRAEDTAVYYCARGAYYGNAMD | |
| | YWGQGTLVTVSSGGGGSGGGGSGGGGSGGGGSDIQMTQSPSSLSASVGDRVTIT | |
| | CRSSQSLLNSGNQKNYVTWYQQKPGKAPKLLIYWASFLYSGVPSRFSGSGSGTDF | |
| | TLTIS SLQPEDFATYYCQNDYFYPLTFGQGTKVEIK | |
| 3-P21- | DIQMTQSPSSLSASVGDRVTITCRSSQSLLNSGNQKNYVTWYQQKPGKAPKLLIY | 212 |
| L1(G₄S)₃H2 | WASFLYSGVPSRFSGSGSGTDFTLTISSLQPEDFATYYCQNDYFYPLTFGQGTKVEI | |
| | KGGGGSGGGGSGGGGSEIQLVESGGGLVQPGGSLRLSCAASGYSFTGYNMKWV | |
| | RQAPGKGLEWIGNINPYFGSTNYADSVKGRATLSVDKSKNTAYLQMNSLRAEDT | |
| | AVYYCARGAYYGNAMDYWGQGTLVTVSS | |
| 15-D6- | EVQLVESGGGLVQPGGSLRLSCAASGYTFTSYWINWVRQAPGKGLEWIGYIYPG | 213 |
| H5(G₄S)₃L4 | RSSTNYNEKFKGRATLSVDTSKNTAYLQMNSLRAEDTAVYYCSRLSRGNAMDY | |
| | WGQGTLVTVSSGGGGSGGGGSGGGGSDIQMTQSPSSLSASVGDRVTITCRSSQSL | |
| | LNSGNQKSYVTWYQQKPGKAPKLLIYWASHRYTGVPSRFSGSGSGTDFTLTISSL | |
| | QPEDFATYYCQNDYYYPFTFGQGTKVEIK | |
| 15-D6- | EVQLVESGGGLVQPGGSLRLSCAASGYTFTSYWINWVRQAPGKGLEWIGYIYPG | 214 |
| H5(G₄S)₄L4 | RSSTNYNEKFKGRATLSVDTSKNTAYLQMNSLRAEDTAVYYCSRLSRGNAMDY | |
| | WGQGTLVTVSSGGGGSGGGGSGGGGSGGGGSDIQMTQSPSSLSASVGDRVTITC | |
| | RSSQSLLNSGNQKSYVTWYQQKPGKAPKLLIYWASHRYTGVPSRFSGSGSGTDFT | |
| | LTISSLQPEDFATYYCQNDYYYPFTFGQGTKVEIK | |
| 15-D6- | DIQMTQSPSSLSASVGDRVTITCRSSQSLLNSGNQKSYVTWYQQKPGKAPKLLIY | 215 |
| L4(G₄S)₃H5 | WASHRYTGVPSRFSGSGSGTDFTLTISSLQPEDFATYYCQNDYYYPFTFGQGTKV | |
| | EIKGGGGSGGGGSGGGGSEVQLVESGGGLVQPGGSLRLSCAASGYTFTSYWINW | |
| | VRQAPGKGLEWIGYIYPGRSSTNYNEKFKGRATLSVDTSKNTAYLQMNSLRAED | |
| | TAVYYCSRLSRGNAMDYWGQGTLVTVSS | |

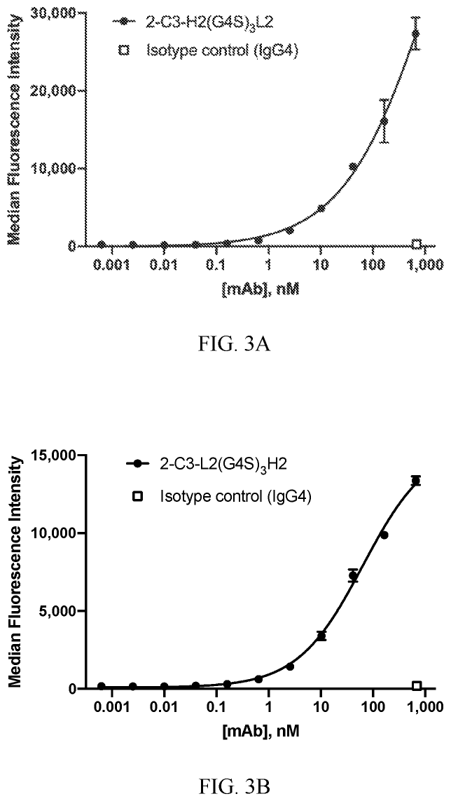

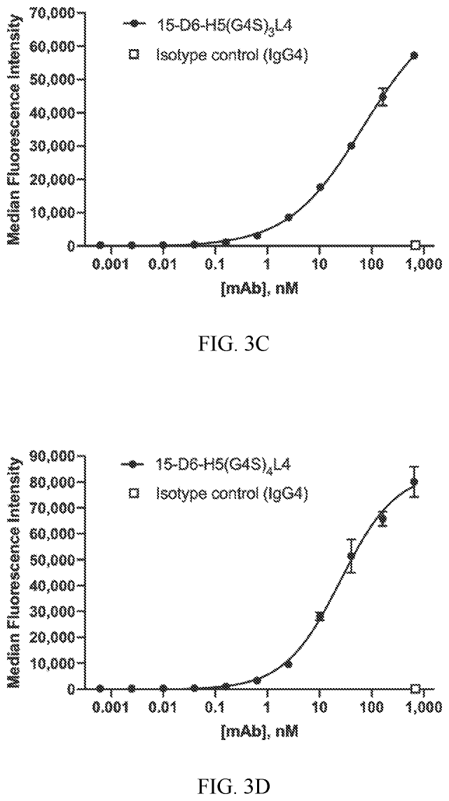

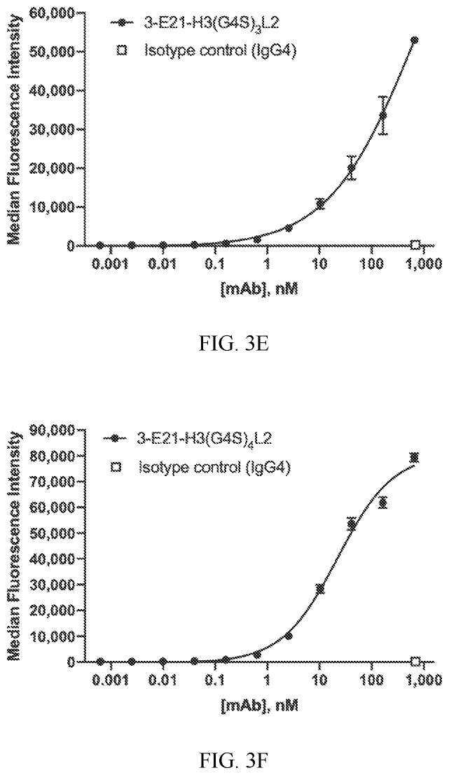

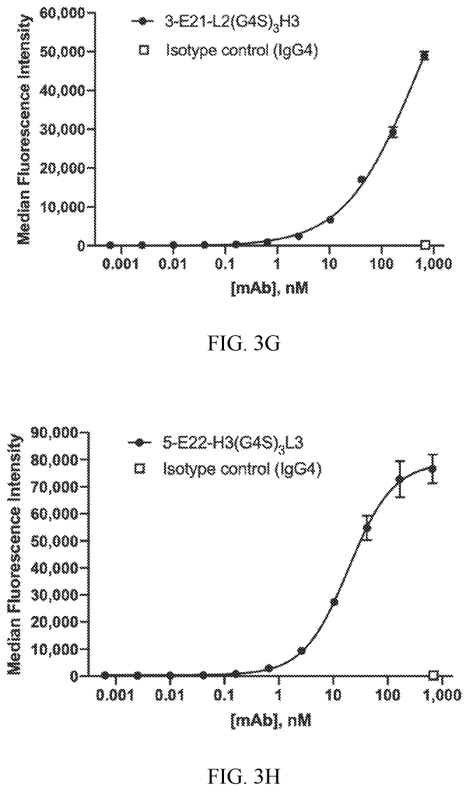

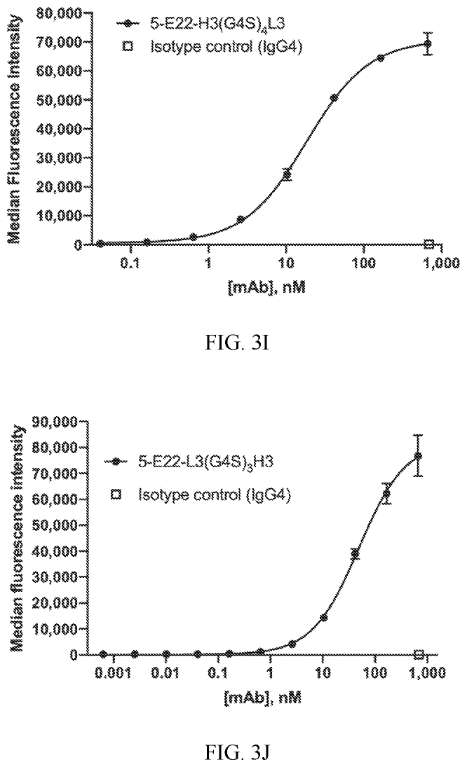

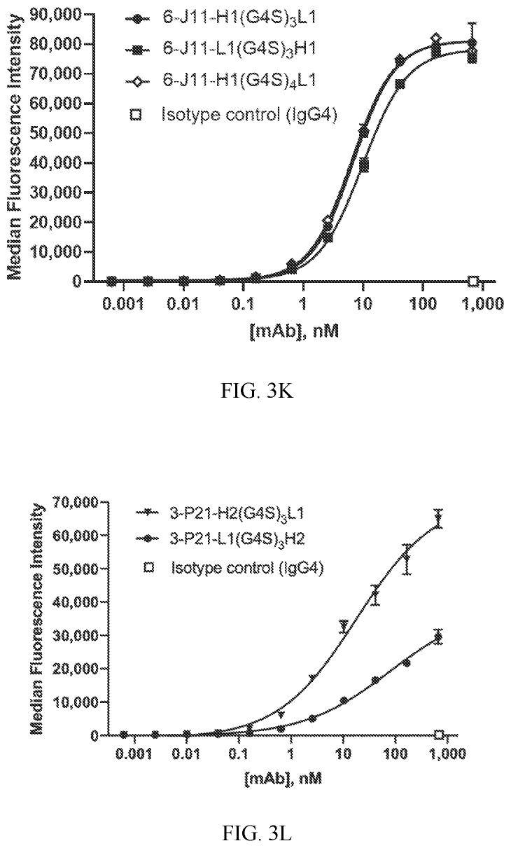

Fusion proteins of scFvs fused to one (G₄S) linker and human IgG4 Fc (with the order of scFv, G₄S linker and Fc from the N-terminus to the C-terminus) were tested for their ability to bind CLDN18.2. A stable cell line (HEK293-CLDN18.2) expressing human CLDN18.2 was used in FACS experiments with Alexa Fluor® 488-based detection as described in PCT/US19/020872. Propidium iodide was incubated together with the secondary antibody to label dead cells. The binding results are shown in FIGS. 3A-3L.

### Example 4: Construction of Chimeric Antigen Receptor Constructs Comprising Anti-CLDN18.2 Antigen Binding Domains

To construct a CAR, the mAbs were converted into scFvs using the VH, VL and a (G₄S)ₙ linker, and the scFv was fused to the N-terminus of the hinge and transmembrane domains derived from human CD8α (aa 114-188, Boursier J P et al., J Biol Chem.

1993;268(3):2013-20). The C-terminal intracellular signaling domain of the CAR was constructed by fusing the intracellular costimulatory domain of CD28 (aa 162-202, Aruffo A and Seed B, Proc Natl Acad Sci USA. 1987;84(23):8573-7) followed by the activation domain from CD3 zeta chain (aa 52-162, Letourneur F and Klausner R D, Proc Natl Acad Sci USA. 1991;88(20):8905-9). The DNA sequence encoding the CAR was assembled and cloned into an expression vector (either retroviral, lentiviral, extrachromosomal or integrated) to generate the CAR construct using standard molecular biology cloning techniques.

### Example 5: Tumor Cell Killing Assay to Assess the Activity of CAR T Cells

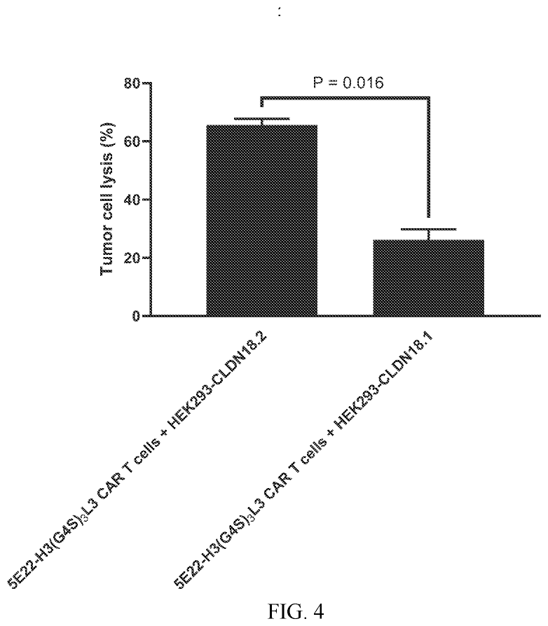

CD4+/CD8+ T cells were isolated using the Pan T isolation kit (Miltenyi biotech, Cat#: 130-096-535), and activated for 3 days by Dynabeads™ Human T-Activator CD3/CD28 (ThermoFisher, Cat#: 11131D) in AIM V medium (ThermoFisher, Cat#: 12055083) containing 10% FBS according to the manufacture instructions. Next, active T cells were continuously cultured for less than a week in AIM V medium containing 10% FBS and 300 IU/ml IL2 (R&D systems, Cat#: 202-IL-050) and transiently transfected with the 5E22-H3(G4S)₃L3 CAR expression plasmid by electroporation to obtain the CAR T cells. Following a 48-hour recovery period, the CAR T cells were used in the assay as the effector cells. Target cells HEK293-CLDN1 8.2 and HEK293-CLDN18.1 were stained with CFSE (ThermoFisher, Cat#: C34554) and co-cultured with the CAR T cells for 24 hours at the E/T (effector/target) ratio of 2.5:1. Next, the cells were stained with PI (ThermoFisher, Cat#: P3566) and Annexin V (Biolegend, Cat#: 640924) and analyzed by flow cytometry (Attune NxT). Only CFSE positive cells were counted. The tumor cell lysis percentages were calculated as the percentage of PI and/or Annexin V positive cells and are shown in FIG. 4.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated polynucleotide comprising a nucleic acid sequence encoding a chimeric antigen receptor (CAR), wherein the CAR comprises:
(a) an extracellular domain comprising at least one antigen binding domain that specifically binds claudin 18.2 (CLDN18.2);
(b) a hinge region;
(c) a transmembrane region; and
(d) an intracellular signaling domain.

2. The isolated polynucleotide of claim 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 21, 22, 23, 51, 52 and 53, respectively, or SEQ ID NOs: 81, 82, 83, 111, 112 and 113, respectively;
(2) SEQ ID NOs: 24, 25, 26, 54, 55 and 56, respectively, or SEQ ID NOs: 84, 85, 86, 114, 115 and 116, respectively;
(3) SEQ ID NOs: 27, 28, 29, 57, 58 and 59, respectively, or SEQ ID NOs: 87, 88, 89, 117, 118 and 119, respectively;
(4) SEQ ID NOs: 30, 31, 32, 60, 61 and 62, respectively, or SEQ ID NOs: 90, 91, 92, 120, 121 and 122, respectively;
(5) SEQ ID NOs: 33, 34, 35, 63, 64 and 65, respectively, or SEQ ID NOs: 93, 94, 95, 123, 124 and 125, respectively;
(6) SEQ ID NOs: 36, 37, 38, 66, 67 and 68, respectively, or SEQ ID NOs: 96, 97, 98, 126, 127 and 128, respectively;
(7) SEQ ID NOs: 39, 40, 41, 69, 70 and 71, respectively, or SEQ ID NOs: 99, 100, 101, 129, 130 and 131, respectively;
(8) SEQ ID NOs: 42, 43, 44, 72, 73 and 74, respectively, or SEQ ID NOs: 102, 103, 104, 132, 133 and 134, respectively;
(9) SEQ ID NOs: 45, 46, 47, 75, 76 and 77, respectively, or SEQ ID NOs: 105, 106, 107, 135, 136 and 137, respectively; or
(10) SEQ ID NOs: 48, 49, 50, 78, 79 and 80, respectively, or SEQ ID NOs: 108, 109, 110, 138, 139 and 140, respectively.

3. (canceled)

4. The isolated polynucleotide of claim 1, wherein the antigen binding domain comprises a heavy chain variable region having a polypeptide sequence at least 95% identical to SEQ ID NO: 1, 3, 5, 7, 9, 11, 13, 15, 17, 19, 142, 143, 146, 147, 151, 152, 154, 155, 156, 159, 160, 161, 162, 166, 167, 170, 171, 172, 175, 176, 177, 178, 179, 180, 186, 187, 191, 192, or 193, or a light chain variable region having a polypeptide sequence at least 95% identical to SEQ ID NO: 2, 4, 6, 8, 10, 12, 14, 16, 18, 20, 144, 145, 148, 149, 150, 153, 157, 158, 163, 164, 165, 168, 169, 173, 174, 181, 182, 183, 184, 185, 188, 189, 190, 194, 195, 196, or 197.

5. The isolated polynucleotide of claim 1, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18;
(10) a heavy chain variable region having the polypeptide sequence of SEQ ID NO:19, and a light chain variable region having the polypeptide sequence of SEQ ID NO:20;
(11) a heavy chain variable region having the polypeptide sequence of SEQ ID NO:142, and a light chain variable region having the polypeptide sequence of SEQ ID NO:144;
(12) a heavy chain variable region having the polypeptide sequence of SEQ ID NO:142, and a light chain variable region having the polypeptide sequence of SEQ ID NO:145;
(13) a heavy chain variable region having the polypeptide sequence of SEQ ID NO:143, and a light chain variable region having the polypeptide sequence of SEQ ID NO:144;
(14) a heavy chain variable region having the polypeptide sequence of SEQ ID NO:143, and a light chain variable region having the polypeptide sequence of SEQ ID NO:145;
(15) a heavy chain variable region having the polypeptide sequence of SEQ ID NO:146, and a light chain variable region having the polypeptide sequence of SEQ ID NO:148;
(16) a heavy chain variable region having the polypeptide sequence of SEQ ID NO:146, and a light chain variable region having the polypeptide sequence of SEQ ID NO:149;
(17) a heavy chain variable region having the polypeptide sequence of SEQ ID NO:146, and a light chain variable region having the polypeptide sequence of SEQ ID NO:150;
(18) a heavy chain variable region having the polypeptide sequence of SEQ ID NO:147, and a light chain variable region having the polypeptide sequence of SEQ ID NO:148;
(19) a heavy chain variable region having the polypeptide sequence of SEQ ID NO:147, and a light chain variable region having the polypeptide sequence of SEQ ID NO:149;
(20) a heavy chain variable region having the polypeptide sequence of SEQ ID NO:147, and a light chain variable region having the polypeptide sequence of SEQ ID NO:150;
(21) a heavy chain variable region having the polypeptide sequence of SEQ ID NO:151, and a light chain variable region having the polypeptide sequence of SEQ ID NO:153;
(22) a heavy chain variable region having the polypeptide sequence of SEQ ID NO:152, and a light chain variable region having the polypeptide sequence of SEQ ID NO:153;
(23) a heavy chain variable region having the polypeptide sequence of SEQ ID NO:154, and a light chain variable region having the polypeptide sequence of SEQ ID NO:157;
(24) a heavy chain variable region having the polypeptide sequence of SEQ ID NO:155, and a light chain variable region having the polypeptide sequence of SEQ ID NO:157;
(25) a heavy chain variable region having the polypeptide sequence of SEQ ID NO:156, and a light chain variable region having the polypeptide sequence of SEQ ID NO:158;
(26) a heavy chain variable region having the polypeptide sequence of SEQ ID NO:159, and a light chain variable region having the polypeptide sequence of SEQ ID NO:163;
(27) a heavy chain variable region having the polypeptide sequence of SEQ ID NO:159, and a light chain variable region having the polypeptide sequence of SEQ ID NO:164;
(28) a heavy chain variable region having the polypeptide sequence of SEQ ID NO:160, and a light chain variable region having the polypeptide sequence of SEQ ID NO:163;
(29) a heavy chain variable region having the polypeptide sequence of SEQ ID NO:160, and a light chain variable region having the polypeptide sequence of SEQ ID NO: 164;
(30) a heavy chain variable region having the polypeptide sequence of SEQ ID NO:161, and a light chain variable region having the polypeptide sequence of SEQ ID NO:165; or
(31) a heavy chain variable region having the polypeptide sequence of SEQ ID NO:162, and a light chain variable region having the polypeptide sequence of SEQ ID NO:165.

6-7. (canceled)

8. The isolated polynucleotide of claim 1, wherein the antigen binding domain is a single chain variable fragment (scFv).

9. The isolated polynucleotide of claim 8, wherein the single chain variable fragment (scFv) is humanized and comprises a polypeptide sequence at least 95% identical to any one of SEQ ID NOs: 198-215.

10. The isolated polynucleotide of claim 1, wherein the chimeric antigen receptor (CAR) comprises one or more antigen binding domains and/or wherein the intracellular signaling domain comprises one or more costimulatory domains and one or more activating domains.

11. (canceled)

12. A chimeric antigen receptor (CAR) encoded by the isolated polynucleotide of claim 1.

13. A vector comprising the isolated polynucleotide of claim 1.

14. A host cell comprising the vector of claim 13.

15. The host cell of claim 14, wherein the host cell is a T cell or NK cell.

16. (canceled)

17. A method of making a host cell expressing a chimeric antigen receptor (CAR), the method comprising transducing a T cell or NK cell with the vector of claim 13.

18. A method of producing a chimeric antigen receptor (CAR)-T cell or a chimeric antigen receptor (CAR)-NK cell, the method comprising culturing T cells or NK cells comprising the isolated polynucleotide comprising a nucleic acid encoding a chimeric antigen receptor (CAR) of claim 1 under conditions to produce the CAR-T cell or CAR-NK cell and recovering the CAR-T cell or CAR-NK cell.

19-20. (canceled)

21. A method of generating a cell comprising a chimeric antigen receptor (CAR), the method comprising contacting a cell with the isolated polynucleotide comprising a nucleic acid encoding a chimeric antigen receptor (CAR) of claim 1, wherein the isolated polynucleotide is an in vitro transcribed RNA or synthetic RNA.

22. A method of treating cancer or an inflammatory disease in a subject in need thereof, the method comprising administering to the subject the host cell of claim 14.

23. The method of claim 22, wherein the cancer is selected from a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

24. (canceled)

25. The method of claim 22, further comprising administering to the subject in need thereof an agent that increases the efficacy of a cell expressing a CAR, an agent that ameliorates one or more side effects associated with administration of a cell expressing a CAR molecule, or an agent that treats the disease associated with Claudin 18.2.

26-27. (canceled)

28. The isolated polynucleotide of claim 1, wherein the CLDN18.2 is human CLDN18.2.

29. The host cell of claim 15, wherein the T cell or NK cell is a human T cell or human NK cell.
